SEQUENCE LISTING (17 sequences)

SEQ ID NO: 1 - protein, 120 aa, synthetic construct
  source 1..120: note = heavy chain variable region (VH) of the exemplary anti-APRIL antibody
QVQLVQSGAEVKKPGASVKVSCKASGYTFTDYTIHWVRQATGQGLEWMGWIYPLRGSINYAQKFQGRVTMTADKSISTVYMELSSLRSEDTAVYFCARHGAYYSNAFDYWGQGTLVTVSS

SEQ ID NO: 2 - protein, 111 aa, synthetic construct
  source 1..111: note = light chain variable region (VL) of the exemplary anti-APRIL antibody
EIVMTQSPATLSVSPGERATLSCRASESVDNDGIRFLHWYQQKPGQAPRLLIYRASTRATGIPARFSGSGSRTEFTLTISSLQSEDFAVYYCQQSNKDPYTFGGGTKVEIK

SEQ ID NO: 3 - protein, 7 aa, synthetic construct
  source 1..7: note = HCDR1
GYTFTDY

SEQ ID NO: 4 - protein, 6 aa, synthetic construct
  source 1..6: note = HCDR2
YPLRGS

SEQ ID NO: 5 - protein, 11 aa, synthetic construct
  source 1..11: note = HCDR3
HGAYYSNAFDY

SEQ ID NO: 6 - protein, 15 aa, synthetic construct
  source 1..15: note = LCDR1
RASESVDNDGIRFLH

SEQ ID NO: 7 - protein, 7 aa, synthetic construct
  source 1..7: note = LCDR2
RASTRAT

SEQ ID NO: 8 - protein, 9 aa, synthetic construct
  source 1..9: note = LCDR3
QQSNKDPYT

SEQ ID NO: 9 - protein, 446 aa, synthetic construct
  source 1..446: note = Heavy Chain
QVQLVQSGAEVKKPGASVKVSCKASGYTFTDYTIHWVRQATGQGLEWMGWIYPLRGSINYAQKFQGRVTMTADKSISTVYMELSSLRSEDTAVYFCARHGAYYSNAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 218 aa, synthetic construct
  source 1..218: note = Light Chain
EIVMTQSPATLSVSPGERATLSCRASESVDNDGIRFLHWYQQKPGQAPRLLIYRASTRATGIPARFSGSGSRTEFTLTISSLQSEDFAVYYCQQSNKDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 250 aa, Homo sapiens [sequence omitted]

SEQ ID NO: 12 - protein, 448 aa, synthetic construct
  source 1..448: note = 4540 anti-APRIL mAb Heavy Chain
QVQLQQSGPELVKPGASVKISCKASGYTFADYYINWVKQRPGQGLEWIGWIFPGSGSTYYNEKFKGKATLTVDKSSSTAYMLLSSLTSEDSAVYFCARGDSGRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEVDGGPSVFLFPPKPKDTLMISRTPEVTCVVVGVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 213 aa, synthetic construct
  source 1..213: note = 4540 anti-APRIL mAb Light Chain
DIQMTQSPSSLSASLGGKVTITCKASQDINKYIAWYQHKPGKGPRLLIHYTSTLQSGIPSRFSGSGSGRDYSFSISNLEPEDNATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 281 aa, synthetic construct
  source 1..281: note = POV001 (based on Povetacicept)
SLSCRKEQGEYYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 313 aa, synthetic construct
  source 1..313: note = ATA001 (based on Atacicept)
AMRSCPEEQYWDPLLGTCMSCKTICNHQSQRTCAAFCRSLSCRKEQGKFYDHLLRDCISCASICGQHPKQCAYFCENKLRSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 450 aa, synthetic construct
  source 1..450: note = MOTA (based on Motavizumab) heavy chain variable region (VH)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 213 aa, synthetic construct
  source 1..213: note = MOTA (based on Motavizumab) light chain variable region (VL)
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0337287 A1
(43) Pub. Date: Oct. 29, 2026

(54) METHODS OF USING ANTIBODIES TO A PROLIFERATION-INDUCING LIGAND (APRIL)

(71) Applicant: Visterra, Inc., Waltham, MA (US)

(72) Inventors: Zachary SHRIVER, Waltham, MA (US); Gregory BABCOCK, Waltham, MA (US); Luke ROBINSON, Waltham, MA (US); Kirk ROWLEY, Waltham, MA (US); Vinay SINGH, Waltham, MA (US); Katherine HEANG, Waltham, MA (US); Anna ROBERTS, Waltham, MA (US); Prashanna Balaji VENKATASUBRAMANIAN, Waltham, MA (US)

(21) Appl. No.: 19/630,770

(22) Filed: Mar. 27, 2026

Related U.S. Application Data
(60) Provisional application No. 63/779,696, filed on Mar. 28, 2025.
(60) Provisional application No. 63/906,881, filed on Oct. 28, 2025.

(51) Int. Cl. C07K 16/28; A61K 38/00; A61K 39/00; A61K 47/68; A61P 13/12; A61P 31/14; A61P 31/16; A61P 37/06; C07K 14/705

(52) U.S. Cl. CPC C07K 16/2875; A61K 47/6811; A61P 13/12; A61P 31/14; A61P 31/16; A61P 37/06; C07K 14/70578; A61K 38/00; A61K 2039/505; C07K 2317/565; C07K 2317/76; C07K 2319/30

(57) ABSTRACT

Methods of treating IgA nephropathy or autoimmune diseases associated with APRIL in a subject who needs treatment for IgA nephropathy or an autoimmune disease and has a risk of an infection during the treatment by selectively administering the subject an anti-APRIL antibody instead of an APRIL/BAFF inhibitor are disclosed.

### CROSS REFERENCE TO RELATED APPLICATIONS

This application claims the benefit of the filing dates of U.S. Provisional Application No. 63/906,881, filed Oct. 28, 2025, and U.S. Provisional Application No. 63/779,696, filed Mar. 28, 2025, the contents of which are incorporated herein by reference in their entirety.

### SEQUENCE LISTING

The content of the electronically submitted sequence listing (Name: 5414_0130002_SequenceListing_ST26.xml; Size: 22,013 bytes; and Date of Creation: Mar. 24, 2026) is incorporated herein by reference in its entirety.

### BACKGROUND

IgAN is caused by the deposition of IgA, typically in the form of immune complexes in the mesangium of the kidney. APRIL (A PRoliferation Inducing Ligand) plays a role in a number of biological processes such as signal transduction, regulation of cell proliferation, and IgA class switching. APRIL is both functionally and structurally related to BAFF (B Cell Activating Factor F13B). And both APRIL and BAFF are essential cytokines for B cell and plasma cell development and survival. Inhibition of APRIL and BAFF has emerged as a strategy for treating immune-mediated diseases and kidney disorders, such as IgAN, but their broader impacts on immune homeostasis are not fully known.

Given the importance of B cells in immunity, such inhibition may pose risks of immunosuppression and susceptibility to infection; thus there is a present need to determine the broader impacts of APRIL and BAFF inhibition on immune homeostasis. Commercially available BAFF inhibitor labels warn that treatment with BAFF inhibitors should be interrupted if patients develop an infection during treatment with BAFF inhibitors.

### SUMMARY

The present disclosure provides a composition comprising an anti-APRIL therapy suitable for use in treating an IgA nephropathy or autoimmune disease in a subject in need thereof, wherein the use comprises (a) identifying a subject who needs a treatment for an IgA nephropathy or autoimmune disease and has a risk of an infection during the treatment, (b) selecting, among anti-APRIL therapies comprising an anti-APRIL antibody and an APRIL/BAFF inhibitor, an anti-APRIL antibody as the anti-APRIL therapy suitable for the subject, and (c) administering the anti-APRIL antibody to the subject. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

In some aspects, the present disclosure provides a method of providing an anti-APRIL therapy for treating an IgA nephropathy or autoimmune disease in a subject in need thereof (a) identifying a subject who needs a treatment for an IgA nephropathy or autoimmune disease and has a risk of an infection during the treatment, (b) selecting, among anti-APRIL therapies comprising an anti-APRIL antibody and an APRIL/BAFF inhibitor, an anti-APRIL antibody as an anti-APRIL therapy suitable for the subject, and (c) administering the anti-APRIL antibody to the subject. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

In some aspects, the present disclosure provides a method of identifying a subject suitable for the treatment of an anti-APRIL therapy comprising (a) assessing the subject's risk of infection during the treatment and (b) selecting an anti-APRIL antibody as a suitable treatment if the subject has a risk of infection during the treatment. In some aspects, the method further comprises administering to the subject an anti-APRIL antibody. In some aspects, the anti-APRIL therapy comprises an anti-APRIL antibody therapy or an APRIL/BAFF inhibitor therapy. In some aspects, the subject is in need of treatment for an IgA nephropathy or autoimmune disease. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

In some aspects, the present disclosure provides a method of reducing or preventing a risk of infection in a subject in need of an anti-APRIL therapy comprising administering to the subject an anti-APRIL antibody. In some aspects, the anti-APRIL therapy comprises an anti-APRIL antibody therapy or an APRIL/BAFF inhibitor therapy. In some aspects, the subject is in need of treatment for an IgA nephropathy or autoimmune disease. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

In some aspects, the present disclosure provides a method of treating an IgA nephropathy or autoimmune disease in a subject in need thereof comprising administering to the subject an anti-APRIL antibody, wherein the subject is identified as having a risk of infection during the treatment. In some aspects, the subject does not have a severe and/or active infection. In some aspects, the present disclosure provides a method of treating an IgA nephropathy or autoimmune disease in a subject in need thereof comprising administering to the subject an anti-APRIL antibody after the subject has developed an infection. In some aspects, the subject has a mild infection. In some aspects, the subject has a moderate infection. In some aspects, the anti-APRIL antibody or the APRIL inhibitor is administered without treatment interruption or discontinuation.

In some aspects, the subject is administered an APRIL/BAFF inhibitor prior to the anti-APRIL antibody. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

In some aspects, administering does not alter B cell population in the subject. In some aspects, the present disclosure provides a method of maintaining B cell population during an anti-APRIL therapy in a subject in need of the therapy comprising administering to the subject an anti-APRIL antibody. In some aspects, the B cell population comprises total B cells, naïve B cells, T1/T2 B cells, follicular (FO) B cells, marginal zone (MZ) B cells, memory B cells, switched memory B cells, plasmablasts, B-1 cells, or regulatory B (Breg) cells.

In some aspects, administering does not alter the population of T follicular helper (Tfh) cells. In some aspects, administering does not significantly alter the B cell population compared to an APRIL/BAFF inhibitor. In some aspects, the B cell population comprises total B cells, naïve B cells, T1/T2 B cells, follicular (FO) B cells, marginal zone (MZ) B cells, memory B cells, switched memory B cells, plasmablasts, B-1 cells, or regulatory B (Breg) cells. In some aspects, administering reduces IgA. In some aspects, administering does not reduce IgM and/or IgG. In some aspects, the infection comprises a viral infection, a bacterial infection, a fungal infection, or a parasitic infection. In some aspects, the infection causes an infectious disease. In some aspects, the infectious disease comprises nasopharyngitis, Coronavirus disease (COVID-19), influenza, sinusitis, pneumonia, or tuberculosis. In some aspects, the autoimmune disease comprises rheumatoid arthritis, systemic lupus erythematosus, a linear IgA bullous disease, IgA-mediated epidermolysis bullosa acquisita, or Sjögren's disease. In some aspects, the anti-APRIL therapy that is an APRIL/BAFF inhibitor comprises co-administering an APRIL inhibitor and a BAFF inhibitor. In some aspects, the APRIL inhibitor comprises zigakibart/BION-1301 or sibeprenlimab. In some aspects, the BAFF inhibitor comprises belimumab, tabalumab, AMG570, blisibimod, or ianalumab.

In some aspects, the present disclosure provides a method of treating autoimmune disease in a subject in need thereof, comprising administering to the subject an APRIL/BAFF inhibitor, wherein the method reduces the B cell population. In some aspects, the B cell population comprises total B cells, naïve B cells, T1/T2 B cells, follicular (FO) B cells, marginal zone (MZ) B cells, memory B cells, switched memory B cells, plasmablasts, B-1 cells, or regulatory B (Breg) cells. In some aspects, the method reduces the population of T follicular helper (Tfh) cells. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the autoimmune disease comprises rheumatoid arthritis, systemic lupus erythematosus, a linear IgA bullous disease, IgA-mediated epidermolysis bullosa acquisita, or Sjögren's disease.

In some aspects, the present disclosure provides a pharmaceutical composition comprising: (a) an anti-APRIL antibody and (b) one or more ingredients comprising L-arginine, L-glutamic acid, L-histidine, L-histidine monohydrochloride, polysorbate 80, and/or sorbitol. In some aspects the present disclosure provides a pharmaceutical composition comprising: (a) an anti-APRIL antibody and (b) one or more ingredients comprising L-arginine, L-glutamic acid, L-histidine, polysorbate 80, and/or sorbitol.

In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301.

In some aspects, the pharmaceutical composition is a liquid formulation. In some aspects, the pharmaceutical composition is a liquid formulation for subcutaneous injection. In some aspects, the pharmaceutical composition is contained within a syringe. In some aspects, the pharmaceutical composition is contained within an autoinjector. In some aspects, the pH of the pharmaceutical composition is between about 5 and about 7, e.g., about 5.0, about 5.25, about 5.5, about 5.75, about 6.0, about 6.25, about 6.5, about 6.75, or about 7.0. In some aspects, the pH is about 6.2. In some aspects, the total volume of the composition is between about 1 and about 3 mL, e.g., about 1 mL, about 1.25 mL, about 1.5 mL, about 1.75 mL, about 2.0 mL, about 2.25 mL, about 2.5 mL, about 2.75 mL, or about 3.0 mL. In some aspects, the total volume of the composition is about 2 mL. In some aspects, the pharmaceutical composition comprises about 400 mg of the anti-APRIL antibody, about 17.6 mg of L-arginine, about 14.8 mg of L-glutamic acid, about 4.3 mg of L-histidine, about 4.6 mg of L-histidine monohydrochloride, about 0.4 mg of polysorbate 80, and about 36.4 mg of sorbitol. In some aspects, the pharmaceutical composition further comprises water for injection. In some aspects, the pH of the composition is about 6.2. In some aspects, the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol. In some aspects, the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol, and wherein the pH of the composition is about 6.2. In some aspects, the composition comprises about 200 mg/mL of the anti-APRIL antibody, about 51 mM L-arginine, about 50 mM L-glutamic acid, about 25 mM L-histidine, about 200 mg/mL polysorbate 80, and about 100 mM sorbitol. In some aspects, the pharmaceutical composition further comprises a trace amount of a chloride ion. In some aspects, the composition comprises between about 10 mg and about 1000 mg of the anti-APRIL antibody, e.g., about 10 mg, about 50 mg, about 100 mg, about 150 mg, about 200 mg, about 250 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 800 mg, about 900 mg, or about 1000 mg. In some aspects, the composition comprises about 400 mg of the anti-APRIL antibody. In some aspects, the pharmaceutical composition further comprises an overfill. In some aspects, the overfill comprises about 0.0125 mL to about 0.5 mL. In some aspects, the overfill comprises about 0.025 mL to about 0.5 mL, about 0.05 mL to about 0.075 mL, about 0.075 mL to about 0.1 mL, about 0.1 mL to about 0.125 mL, about 0.125 mL to about 0.15 mL, about 0.15 mL to about 0.175 mL, about 0.175 mL to about 0.2 mL, about 0.2 mL to about 0.225 mL, about 0.225 mL to about 0.25 mL, about 0.25 mL to about 0.275 mL, about 0.275 mL to about 0.3 mL, about 0.3 mL to about 0.325 mL, about 0.325 mL to about 0.35 mL, about 0.35 mL to about 0.375 mL, about 0.375 mL to about 0.4 mL, about 0.4 mL to about 0.425 mL, about 0.425 mL to about 0.45 mL, about 0.45 mL to about 0.475 mL, or about 0.475 mL to about 0.5 mL. In some aspects, the overfill comprises about 0.0125 mL, 0.025 mL, about 0.05 mL, about 0.075 mL, about 0.1 mL, about 0.125 mL, about 0.15 mL, about 0.175 mL, about 0.2 mL, about 0.225 mL, about 0.25 mL, about 0.275 mL, about 0.3 mL, about 0.325 mL, about 0.35 mL, about 0.375 mL, about 0.4 mL, about 0.425 mL, about 0.45 mL, about 0.475 mL, or about 0.5 mL.

In some aspects, the present disclosure provides a pharmaceutical composition comprising about 51 mM L-arginine, about 50 mM L-glutamic acid, about 14 mM L-histidine, about 11 mM L-histidine monohydrochloride, about 200 mg/mL polysorbate 80, about 100 mM sorbitol, and about 200 mg/mL of the anti-APRIL antibody, wherein the pH of the composition is about 6.2, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.

In some aspects, the present disclosure provides a pharmaceutical composition comprising about 51 mM L-arginine, about 50 mM L-glutamic acid, about 25 mM L-histidine, about 200 mg/mL polysorbate 80, about 100 mM sorbitol, and about 200 mg/mL of the anti-APRIL antibody, wherein the pH of the composition is about 6.2, wherein the total volume of the composition is about 2 mL, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody in the composition is about 400 mg. In some aspects, the anti-APRIL antibody comprises Sibeprenlimab or an antigen binding portion thereof.

In some aspects, the present disclosure provides a method of treating a disease or disorder associated with APRIL, e.g., IgA nephropathy or Sjögren's disease, in a subject in need thereof comprising administering to the subject the pharmaceutical composition.

In some aspects, the present disclosure provides a method of producing a pharmaceutical composition comprising admixing one or more ingredients in the pharmaceutical composition.

Other features, objects, and advantages of the compositions and methods herein will be apparent from the description and drawings, and from the claims.

### EXEMPLARY ASPECTS

[A1] A composition comprising an anti-APRIL therapy suitable for use in treating an IgA nephropathy or autoimmune disease in a subject in need thereof, wherein the use comprises (a) identifying a subject who needs a treatment for an IgA nephropathy or autoimmune disease and has a risk of an infection during the treatment, (b) selecting, among anti-APRIL therapies comprising an anti-APRIL antibody and an APRIL/BAFF inhibitor, an anti-APRIL antibody as the anti-APRIL therapy suitable for the subject, and (c) administering the anti-APRIL antibody to the subject.
[A2] The composition of A1, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8.
[A3] The composition of A1 or A2, wherein the VH comprises the amino acid sequence as set forth in SEQ ID NO: 1.
[A4] The composition of any one of A1-3, wherein the VL comprises the amino acid sequence as set forth in SEQ ID NO: 2.
[A5] The composition of any one of A1-4, wherein the anti-APRIL antibody further comprises a constant region linked to the VH and or VL.
[A6] The composition of any one of A1-5, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A7] The composition of any one of A1, wherein the anti-APRIL antibody comprises a heavy chain (HC) comprising the amino acid sequence as set forth in SEQ ID NO: 12 and a light chain (LC) comprising the amino acid sequence as set forth in SEQ ID NO: 13.
[A8] The composition of A1, wherein the anti-APRIL antibody comprises zigakibart/BION-1301.
[A9] The composition of any one of any one of A1-8, wherein the APRIL/BAFF inhibitor comprises a fusion protein.
[A10] The composition of any one of A1-9, wherein the APRIL/BAFF inhibitor comprises, atacicept, povetacicept, or telitacicept.
[A11] The composition of any one of A1-10, wherein the APRIL/BAFF inhibitor comprises atacicept.
[A12] The composition of any one of A1-11, wherein the APRIL/BAFF inhibitor comprises povetacicept.
[A13] The composition of any one of A1-12, wherein the APRIL/BAFF inhibitor comprises telitacicept.
[A14] The composition of any one of A1-13, wherein the anti-APRIL antibody is administered subcutaneously.
[A15] The composition of any one of A1-14, wherein the anti-APRIL antibody is administered at a fixed dose between 10 mg and 1000 mg.
[A16] The composition of any one of A1-15, wherein the anti-APRIL antibody is administered at a fixed dose of 400 mg once every four weeks.
[A17] The composition of any one of A1-16, wherein the anti-APRIL antibody or the APRIL inhibitor is administered twice, three times, four times, five times, six times, seven times, eight times, nine times, 10 times, 11 times, 12 times, 13 times, 14 times, or 15 times.
[A18] The composition of any one of A1-17, wherein the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks.
[A19] The composition of any one of A1-18, wherein the subject does not have a severe and/or active infection.
[A20] The composition of any one of A1-18, wherein the subject has a mild infection.
[A21] The composition of any one of A1-18, wherein the subject has a moderate infection.
[A22] The composition of any one of A1-21, wherein the anti-APRIL antibody or the APRIL inhibitor is administered without treatment interruption or discontinuation.
[A23] A method of providing an anti-APRIL therapy for treating an IgA nephropathy or autoimmune disease in a subject in need thereof (a) identifying a subject who needs a treatment for an IgA nephropathy or autoimmune disease and has a risk of an infection during the treatment, (b) selecting, among anti-APRIL therapies comprising an anti-APRIL antibody and an APRIL/BAFF inhibitor, an anti-APRIL antibody as an anti-APRIL therapy suitable for the subject, and (c) administering the anti-APRIL antibody to the subject.
[A24] The method of A23, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8
[A25] The method of A23 or 24, wherein the VH comprises the amino acid sequence as set forth in SEQ ID NO: 1.
[A26] The method of any one of A23-25, wherein the VL comprises the amino acid sequence as set forth in SEQ ID NO: 2.
[A27] The method of any one of A23-26, wherein the anti-APRIL antibody further comprises a constant region linked to the VH and or VL.
[A28] The method of any one of 23-27, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A29] The method of A23, wherein the anti-APRIL antibody comprises a heavy chain (HC) comprising the amino acid sequence as set forth in SEQ ID NO: 12 and a light chain (LC) comprising the amino acid sequence as set forth in SEQ ID NO: 13.
[A30] The method of A23, wherein the anti-APRIL antibody comprises zigakibart/BION-1301.
[A31] The method of any one of any one of A23-30, wherein the APRIL/BAFF inhibitor comprises a fusion protein.
[A32] The method of any one of A23-31, wherein the APRIL/BAFF inhibitor comprises, atacicept, povetacicept, or telitacicept.
[A33] The method of any one of A23-32, wherein the APRIL/BAFF inhibitor comprises atacicept.
[A34] The method of any one of A23-33, wherein the APRIL/BAFF inhibitor comprises povetacicept.
[A35] The method of any one of A23-34, wherein the APRIL/BAFF inhibitor comprises telitacicept.
[A36] The method of any one of any one of A23-35, wherein the anti-APRIL antibody is administered subcutaneously.
[A37] The method of any one of any one of A23-36, wherein the anti-APRIL antibody is administered at a fixed dose between 10 mg and 1000 mg.
[A38] The method of any one of any one of A23-37, wherein the anti-APRIL antibody is administered at a fixed dose of 400 mg once every four weeks.
[A39] The method of any one of any one of A23-38, wherein the anti-APRIL antibody or the APRIL inhibitor is administered twice, three times, four times, five times, six times, seven times, eight times, nine times, 10 times, 11 times, 12 times, 13 times, 14 times, or 15 times.
[A40] The method of any one of any one of A23-39, wherein the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks.
[A41] The method of any one of any one of A23-40, wherein the subject does not have a severe and/or active infection.
[A42] The method of any one of any one of A23-41, wherein the subject has a mild infection.
[A43] The method of any one of any one of A23-42, wherein the subject has a moderate infection.
[A44] The method of any one of any one of A23-43, wherein the anti-APRIL antibody or the APRIL inhibitor is administered without treatment interruption or discontinuation.
[A45] A method of identifying a subject suitable for the treatment of an anti-APRIL therapy comprising (a) assessing the subject's risk of infection during the treatment and (b) selecting an anti-APRIL antibody as a suitable treatment if the subject has a risk of infection during the treatment.
[A46] The method of A45, further comprising administering to the subject an anti-APRIL antibody.
[A47] The method of A45 or 46, wherein the anti-APRIL therapy comprises an anti-APRIL antibody therapy or an APRIL/BAFF inhibitor therapy.
[A48] The method of any one of A46-47, wherein the subject is in need of treatment for an IgA nephropathy or autoimmune disease.
[A49] The method of any one of A45-48, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8.
[A50] The method of any one of A45-49, wherein the VH comprises the amino acid sequence as set forth in SEQ ID NO: 1.
[A51] The method of any one of A45-50, wherein the VL comprises the amino acid sequence as set forth in SEQ ID NO: 2.
[A52] The method of any one of A45-51, wherein the anti-APRIL antibody further comprises a constant region linked to the VH and or VL.
[A53] The method of any one of A45-52, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A54] The method of A45, wherein the anti-APRIL antibody comprises a heavy chain (HC) comprising the amino acid sequence as set forth in SEQ ID NO: 12 and a light chain (LC) comprising the amino acid sequence as set forth in SEQ ID NO: 13.
[A55] The method of any one A45-48 wherein the anti-APRIL antibody therapy comprises zigakibart/BION-1301.
[A56] The method of A45, wherein the anti-APRIL antibody comprises zigakibart/BION-1301.
[A57] The method of any one of any one of A45-56, wherein the APRIL/BAFF inhibitor comprises a fusion protein.
[A58] The method of any one of A45-57, wherein the APRIL/BAFF inhibitor comprises, atacicept, povetacicept, or telitacicept.
[A59] The method of any one of A45-58, wherein the APRIL/BAFF inhibitor comprises atacicept.
[A60] The method of any one of A45-59, wherein the APRIL/BAFF inhibitor comprises povetacicept.
[A61] The method of any one of A45-60, wherein the APRIL/BAFF inhibitor comprises telitacicept.
[A62] The method of any one of any one of A45-61, wherein the anti-APRIL antibody is administered subcutaneously.
[A63] The method of any one of any one of A45-62, wherein the anti-APRIL antibody is administered at a fixed dose between 10 mg and 1000 mg.
[A64] The method of any one of any one of A45-63, wherein the anti-APRIL antibody is administered at a fixed dose of 400 mg once every four weeks.
[A65] The method of any one of any one of A45-64, wherein the anti-APRIL antibody or the APRIL inhibitor is administered twice, three times, four times, five times, six times, seven times, eight times, nine times, 10 times, 11 times, 12 times, 13 times, 14 times, or 15 times.
[A66] The method of any one of any one of A45-65, wherein the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks.
[A67] The method of any one of any one of A45-66, wherein the subject does not have a severe and/or active infection.
[A68] The method of any one of any one of A45-67, wherein the subject has a mild infection.
[A69] The method of any one of any one of A45-68, wherein the subject has a moderate infection.
[A70] The method of any one of any one of A45-69, wherein the anti-APRIL antibody or the APRIL inhibitor is administered without treatment interruption or discontinuation.
[A71] A method of reducing or preventing a risk of infection in a subject in need of an anti-APRIL therapy comprising administering to the subject an anti-APRIL antibody.
[A72] The method of A71, wherein the anti-APRIL therapy comprises an anti-APRIL antibody therapy or an APRIL/BAFF inhibitor therapy.
[A73] The method of A71 or 72, wherein the subject is in need of treatment for an IgA nephropathy or autoimmune disease.
[A74] The method of any one of A71-73, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8.
[A75] The method of any one of A71-74, wherein the VH comprises the amino acid sequence as set forth in SEQ ID NO: 1.
[A76] The method of any one of A71-75, wherein the VL comprises the amino acid sequence as set forth in SEQ ID NO: 2.
[A77] The method of any one of A71-76, wherein the anti-APRIL antibody further comprises a constant region linked to the VH and or VL.
[A78] The method of any one of A71-77, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A79] The method of A71, wherein the anti-APRIL antibody comprises a heavy chain (HC) comprising the amino acid sequence as set forth in SEQ ID NO: 12 and a light chain (LC) comprising the amino acid sequence as set forth in SEQ ID NO: 13.
[A80] The method of any one of any one of A71-73, wherein the anti-APRIL antibody therapy comprises zigakibart/BION-1301.
[A81] The method of A71, wherein the anti-APRIL antibody comprises zigakibart/BION-1301.
[A82] The method of any one of any one of A71-81, wherein the APRIL/BAFF inhibitor comprises a fusion protein.
[A83] The method of any one of A71-82, wherein the APRIL/BAFF inhibitor comprises, atacicept, povetacicept, or telitacicept.
[A84] The method of A71-83, wherein the APRIL/BAFF inhibitor comprises atacicept.
[A85] The method of A71-84, wherein the APRIL/BAFF inhibitor comprises povetacicept.
[A86] The method of A71-85, wherein the APRIL/BAFF inhibitor comprises telitacicept.
[A87] The method of any one of A71-86, wherein the anti-APRIL antibody is administered subcutaneously.
[A88] The method of any one of A71-87, wherein the anti-APRIL antibody is administered at a fixed dose between 10 mg and 1000 mg.
[A89] The method of any one of A71-88, wherein the anti-APRIL antibody is administered at a fixed dose of 400 mg once every four weeks.
[A90] The method of any one of A71-89, wherein the anti-APRIL antibody or the APRIL inhibitor is administered twice, three times, four times, five times, six times, seven times, eight times, nine times, 10 times, 11 times, 12 times, 13 times, 14 times, or 15 times.
[A91] The method of any one of A71-90, wherein the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks.
[A92] The method of any one of A71-91, wherein the subject does not have a severe and/or active infection.
[A93] The method of any one of A71-91, wherein the subject has a mild infection.
[A94] The method of any one of A71-91, wherein the subject has a moderate infection.
[A95] The method of any one of A71-91, wherein the anti-APRIL antibody or the APRIL inhibitor is administered without treatment interruption or discontinuation.
[A96] A method of treating an IgA nephropathy or autoimmune disease in a subject in need thereof comprising administering to the subject an anti-APRIL antibody, wherein the subject is identified as having a risk of infection during the treatment.
[A97] A method of treating an IgA nephropathy or autoimmune disease in a subject in need thereof comprising administering to the subject an anti-APRIL antibody after the subject has developed an infection.
[A98] The method of A96 or A97, wherein the subject is administered an APRIL/BAFF inhibitor prior to the anti-APRIL antibody.
[A99] The method of any one of A96-98, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8.
[A100] The method of any one of A96-99, wherein the VH comprises the amino acid sequence as set forth in SEQ ID NO: 1.
[A101] The method of any one of A96-100, wherein the VL comprises the amino acid sequence as set forth in SEQ ID NO: 2.
[A102] The method of any one of any one of A96-101, wherein the anti-APRIL antibody further comprises a constant region linked to the VH and or VL.
[A103] The method of any one of A96-102, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A104] The method of A96, wherein the anti-APRIL antibody comprises a heavy chain (HC) comprising the amino acid sequence as set forth in SEQ ID NO: 12 and a light chain (LC) comprising the amino acid sequence as set forth in SEQ ID NO: 13.
[A105] The method of A96, wherein the anti-APRIL antibody comprises zigakibart/BION-1301.
[A106] The method of any one of A96-105, wherein the APRIL/BAFF inhibitor comprises a fusion protein.
[A107] The method of any one of A96-106, wherein the APRIL/BAFF inhibitor comprises, atacicept, povetacicept, or telitacicept.
[A108] The method of any one of A96-107, wherein the APRIL/BAFF inhibitor comprises atacicept.
[A109] The method of any one of A96-108, wherein the APRIL/BAFF inhibitor comprises povetacicept.
[A110] The method of any one of A96-109, wherein the APRIL/BAFF inhibitor comprises telitacicept.
[A111] The method of any one of A96-110, wherein the anti-APRIL antibody is administered subcutaneously. [A112] The method of any one of A96-111, wherein the anti-APRIL antibody is administered at a fixed dose between 10 mg and 1000 mg. [A113] The method of any one of A96-112, wherein the anti-APRIL antibody is administered at a fixed dose of 400 mg once every four weeks.
[A114] The method of any one of A96-113, wherein the anti-APRIL antibody or the APRIL inhibitor is administered twice, three times, four times, five times, six times, seven times, eight times, nine times, 10 times, 11 times, 12 times, 13 times, 14 times, or 15 times.
[A115] The method of any one of A96-114, wherein the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks.
[A116] The method of any one of A96-115, wherein the subject does not have a severe and/or active infection.
[A117] The method of any one of A96-115, wherein the subject has a mild infection.
[A118] The method of any one of A96-115, wherein the subject has a moderate infection.
[A119] The method of any one of A96-118, wherein the anti-APRIL antibody or the APRIL inhibitor is administered without treatment interruption or discontinuation.
[A120] The composition for use of any one of A1-22 or the method of any one of A23-119, wherein the administering does not alter B cell population in the subject.
[A121] The composition for use of any one of A1-22 or the method of any one of A23-119, wherein the administering preserves the B cell population in the subject.
[A122] A method of maintaining B cell population during an anti-APRIL therapy in a subject in need of the therapy comprising administering to the subject an anti-APRIL antibody.
[A123] The composition for use of A120-121 or the method of any one of A120-122, wherein the B cell population comprises total B cells, naïve B cells, T1/T2 B cells, follicular (FO) B cells, marginal zone (MZ) B cells, memory B cells, switched memory B cells, plasmablasts, B-1 cells, or regulatory B (Breg) cells.
[A124] The composition for use of A123 or the method of A123, wherein the B cell population is not altered in the spleen, bone marrow, and/or mesenteric lymph nodes.
[A125] The composition for use of A123 or the method of A123, wherein the B cell population is preserved in the spleen, bone marrow, and/or mesenteric lymph nodes.
[A126] The composition for use of any one of A1-22, A121, or A123-125, or the method of any one of A23-125, wherein the administering does not alter the population of T follicular helper (Tfh) cells.
[A127] The composition for use of any one of A1-22, A121, or A123-125, or the method of any one of A23-125, wherein the administering preserves the population of T follicular helper (Tfh) cells.
[A128] The composition for use of A126 or the method of A126, wherein the population of T follicular helper (Tfh) cells is altered by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%.
[A129] The composition for use of A127 or the method of A127, wherein the population of T follicular helper (Tfh) cells is preserved by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, or about 90%.
[A130] The composition for use of any one of A1-22, A120, A122-124, or A126-130, or the method of any one of A23-120, A122-124, or A126-130, wherein the administering does not alter the B cell population compared to an APRIL/BAFF inhibitor.
[A131] The composition for use of any one of A1-22, A121, A123, or A125-129, or the method of any one of A23-119, A122-123, A125-129, wherein the administering preserves the B cell population compared to an APRIL/BAFF inhibitor.
[A132] The composition for use of any one of A1-22, A120, A122-124, or A126-130 or the method of any one of A23-120, A122-124, or A126-130, wherein the B cell population is altered by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%.
[A133] The composition for use of any one of A1-22, A121, A123, or A125-129 or the method of any one of A23-119, A122-123, A125-129, wherein the B cell population is preserved by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, or about 90%.
[A134] The composition for use of any one of A1-22, A120-133, or the method of any one of A23-133, wherein the administering reduces germinal center B cell population.
[A135] The composition for use of any one of A1-22, A120-134, or the method of any one of A23-134, wherein the administering reduces IgA.
[A136] The composition for use of any one of A1-22, A120-135, or the method of any one of A23-135, wherein the administering does not reduce IgM and/or IgG.
[A137] The composition for use of A136 or the method for use of A136, wherein IgM is reduced by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 20%, about 30%, about 40%, about 50%, about 55%, about 56%, about 57%, about 58%, or about 59%.
[A138] The composition for use of A136 or the method for use of A136, wherein IgG is reduced by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 16%, about 17%, about 18%, about 19%, about 20%, about 25%, about 30%, about 35%, about 40%, about 45%, or about 50%.
[A139] The composition for use of any one of A1-22, A120-138, or the method of any one of A23-138, wherein the infection comprises a viral infection, a bacterial infection, a fungal infection, or a parasitic infection.
[A140] The composition for use of any one of A1-22, A120-139, or the method of any one of A23-139, wherein the infection causes an infectious disease.
[A141] The composition for use of any one of A1-22, A120-140, or the method of any one of A23-140, wherein the infectious disease comprises nasopharyngitis, Coronavirus disease (COVID-19), influenza, sinusitis, pneumonia, or tuberculosis.
[A142] The composition for use of any one of A1-22, A120-141, or the method of any one of A23-141, wherein the autoimmune disease comprises rheumatoid arthritis, systemic lupus erythematosus, a linear IgA bullous disease, IgA-mediated epidermolysis bullosa acquisita, or Sjögren's disease.
[A143] The composition for use of any one of A1-22, A120-141, or the method of any one of A23-141, wherein the anti-APRIL therapy that is an APRIL/BAFF inhibitor comprises co-administering an APRIL inhibitor and a BAFF inhibitor.
[A144] The composition for use of A143 or the method of A143, wherein the APRIL inhibitor comprises zigakibart/BION-1301 or sibeprenlimab.
[A145] The composition for use of A143 or A144, or the method of A143 or A144, wherein the BAFF inhibitor comprises belimumab, tabalumab, AMG570, blisibimod, ianalumab, or Sandy-2.
[A146] A pharmaceutical composition comprising: (a) an anti-APRIL antibody and (b) one or more ingredients comprising L-arginine, L-glutamic acid, L-histidine, L-histidine monohydrochloride, polysorbate 80, and/or sorbitol.
[A147] A pharmaceutical composition comprising: (a) an anti-APRIL antibody and (b) one or more ingredients comprising L-arginine, L-glutamic acid, L-histidine, polysorbate 80, and/or sorbitol.
[A148] The pharmaceutical composition of any one of A135-A136, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8.
[A149] The pharmaceutical composition of any one of A146-148, wherein the VH comprises the amino acid sequence as set forth in SEQ ID NO: 1.
[A150] The pharmaceutical composition of any one of A146-149, wherein the VL comprises the amino acid sequence as set forth in SEQ ID NO: 2.
[A151] The pharmaceutical composition of any one of A146-150, wherein the anti-APRIL antibody further comprises a constant region linked to the VH and or VL.
[A152] The pharmaceutical composition of any one of any one of A146-151, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A153] The pharmaceutical composition of any one of A146 or A147, wherein the anti-APRIL antibody comprises a heavy chain (HC) comprising the amino acid sequence as set forth in SEQ ID NO: 12 and a light chain (LC) comprising the amino acid sequence as set forth in SEQ ID NO: 13.
[A154] The pharmaceutical composition of any one of A146 or A147, wherein the anti-APRIL antibody comprises zigakibart/BION-1301.
[A155] The pharmaceutical composition of any one of A146-154, which is a liquid formulation.
[A156] The pharmaceutical composition of any one of A146-155, which is a liquid formulation for subcutaneous injection.
[A157] The pharmaceutical composition of any one of A146-156, which is contained within a syringe.
[A158] The pharmaceutical composition of any one of A146-157, which is contained within an autoinjector.
[A159] The pharmaceutical composition of any one of A146-158, wherein the pH of the pharmaceutical composition is between about 5 and about 7, e.g., about 5.0, about 5.25, about 5.5, about 5.75, about 6.0, about 6.25, about 6.5, about 6.75, or about 7.0.
[A160] The pharmaceutical composition of A148, wherein the pH is about 6.2.
[A161] The pharmaceutical composition of any one of A146-160, wherein the total volume of the composition is between about 1 and about 3 mL, e.g., about 1 mL, about 1.25 mL, about 1.5 mL, about 1.75 mL, about 2.0 mL, about 2.25 mL, about 2.5 mL, about 2.75 mL, or about 3.0 mL.
[A162] The pharmaceutical composition of any one of A146-161, wherein the total volume of the composition is about 2 mL.
[A163] The pharmaceutical composition of any one of A146-162, which comprises about 400 mg of the anti-APRIL antibody, about 17.6 mg of L-arginine, about 14.8 mg of L-glutamic acid, about 4.3 mg of L-histidine, about 4.6 mg of L-histidine monohydrochloride, about 0.4 mg of polysorbate 80, and about 36.4 mg of sorbitol.
[A164] The pharmaceutical composition of A163, which further comprises water for injection.
[A165] The pharmaceutical composition of any one of claims A161-164, wherein the pH of the composition is about 6.2.
[A166] The pharmaceutical composition of any one of A146-165, wherein the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol.
[A167] The pharmaceutical composition of any one of A146-166, wherein the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol, and wherein the pH of the composition is about 6.2.
[A168] The pharmaceutical composition of any one of A146-167, wherein the composition comprises about 200 mg/mL of the anti-APRIL antibody, about 51 mM L-arginine, about 50 mM L-glutamic acid, about 25 mM L-histidine, about 200 mg/mL polysorbate 80, and about 100 mM sorbitol.
[A169] The pharmaceutical composition of any one of A146-168, further comprising a trace amount of a chloride ion.
[A170] The pharmaceutical composition of any one of A146-169, wherein the composition comprises between about 10 mg and about 1000 mg of the anti-APRIL antibody, e.g., about 10 mg, about 50 mg, about 100 mg, about 150 mg, about 200 mg, about 250 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 800 mg, about 900 mg, or about 1000 mg.
[A171] The pharmaceutical composition of any one of A146-170, wherein the composition comprises about 400 mg of the anti-APRIL antibody.
[A172] The pharmaceutical composition of any one of A146-171, further comprising an overfill.
[A173] The pharmaceutical composition of A172, wherein the overfill comprises about 0.0125 mL to about 0.5 mL.
[A174] The pharmaceutical composition of any one of A173-173, wherein the overfill comprises about 0.0125 mL to about 0.5 mL, about 0.025 to about 0.05 mL, about 0.05 mL to about 0.075 mL, about 0.075 mL to about 0.1 mL, about 0.1 mL to about 0.125 mL, about 0.125 mL to about 0.15 mL, about 0.15 mL to about 0.175 mL, about 0.175 mL to about 0.2 mL, about 0.2 mL to about 0.225 mL, about 0.225 mL to about 0.25 mL, about 0.25 mL to about 0.275 mL, about 0.275 mL to about 0.3 mL, about 0.3 mL to about 0.325 mL, about 0.325 mL to about 0.35 mL, about 0.35 mL to about 0.375 mL, about 0.375 mL to about 0.4 mL, about 0.4 mL to about 0.425 mL, about 0.425 mL to about 0.45 mL, about 0.45 mL to about 0.475 mL, or about 0.475 mL to about 0.5 mL.
[A175] The pharmaceutical composition of any one of A173-174, wherein the overfill comprises about 0.0125 mL, about 0.025 mL, about 0.05 mL, about 0.075 mL, about 0.1 mL, about 0.125 mL, about 0.15 mL, about 0.175 mL, about 0.2 mL, about 0.225 mL, about 0.25 mL, about 0.275 mL, about 0.3 mL, about 0.325 mL, about 0.35 mL, about 0.375 mL, about 0.4 mL, about 0.425 mL, about 0.45 mL, about 0.475 mL, or about 0.5 mL.
[A176] A pharmaceutical composition comprising about 51 mM L-arginine, about 50 mM L-glutamic acid, about 14 mM L-histidine, about 11 mM L-histidine monohydrochloride, about 200 mg/mL polysorbate 80, about 100 mM sorbitol, and about 200 mg/mL of the anti-APRIL antibody, wherein the pH of the composition is about 6.2, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A177] A pharmaceutical composition comprising about 51 mM L-arginine, about 50 mM L-glutamic acid, about 25 mM L-histidine, about 200 mg/mL polysorbate 80, about 100 mM sorbitol, and about 200 mg/mL of the anti-APRIL antibody, wherein the pH of the composition is about 6.2, wherein the total volume of the composition is about 2 mL, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A178] The pharmaceutical composition of A176 or 177, wherein the anti-APRIL antibody in the composition is about 400 mg.
[A179] The pharmaceutical composition of any one of A146-178, wherein the anti-APRIL antibody comprises Sibeprenlimab or an antigen binding portion thereof.
[A180] A method of treating a disease or disorder associated with APRIL, e.g., IgA nephropathy or Sjögren's disease, in a subject in need thereof comprising administering to the subject the pharmaceutical composition of any one of A146-179.
[A181] A method of producing a pharmaceutical composition comprising admixing one or more ingredients in the pharmaceutical composition of any one of A146-179.

### BRIEF DESCRIPTION OF THE DRAWINGS

FIGS. 1A-1C show the abundance and phenotypic changes of B and T cell populations with APRIL or dual APRIL/BAFF inhibition. FIG. 1A shows flow cytometric characterization of the relative frequency of various B- and T-cell populations from mice (n=5/group, except for atacicept (“ATA”) which is n=4) treated with repeated dosing of MOTA (isotype control), 4540 (Anti-APRIL antibody), ATA (TACI-Fc based on VT-001) and povetacicept (“POV”) (TACI-Fc based on ALPN-303). Data represents a comparison to the average number of cells per subtype relative to isotype. Error bars represent standard error to the mean (SEM). *p<0.05, **p<0.01, ***p<0.005, ****p<0.001 values represent one-way ANOVA comparisons to MOTA. FIG. 1B shows flow cytometry contour plots representing changes in CD21/35 expression in mouse splenic follicular B cells. FIG. 1C shows quantitative expression of CD21/35 on mouse splenic follicular B cells, shown as median fluorescence intensity (MFI). Boxes indicate the interquartile range, center lines indicate the median, and whiskers denote the range. Statistical analysis was performed using the Kruskal-Wallis test with Dunn's multiple-comparisons test. Significance is indicated as *p<0.05 and **p<0.01.

FIGS. 2A-2C show serology comparisons of IgA, IgM, and IgG during APRIL or dual APRIL/BAFF inhibition. Data is reported as a relative change to pre-treatment concentrations of immunoglobulin. Error bars represent SEM. A mixed-effect statistical analysis with Geisser-Greenhouse correction was performed for the last two timepoints in comparison to isotype control. *p<0.05, **p<0.01, ***p<0.005, ****p<0.001. FIG. 2A shows serology measurements of IgA from the same animals treated with APRIL or APRIL/BAFF inhibitors as FIGS. 1A-1C were compared to isotype control. FIG. 2B shows serology measurements of IgM from the same animals treated with APRIL or APRIL/BAFF inhibitors as FIGS. 1A-1C were compared to isotype control. FIG. 2C shows serology measurements of IgG from the same animals treated with APRIL or APRIL/BAFF inhibitors as FIGS. 1A-1C were compared to isotype control.

FIGS. 3A-3F depict B-cell subsets (total B cells, FIG. 3A; total T cells, FIG. 3B; naïve B cells, FIG. 3C; memory B cells, FIG. 3D; switched memory B cells, FIG. 3E; and plasmablasts, FIG. 3F) by sibeprenlimab or placebo dose group. Median fold change from baseline in absolute cell counts over time is shown. Error bars±interquartile range are shown. Vertical dashed line indicates end of study treatment period at Day 360 (Month 12).

FIGS. 4A-4B depict the study design for an Influenza challenge of APRIL or APRIL/BAFF Treated C57BL/6 mice and Kaplan-Meier survival curves for each study design group. FIG. 4A depicts the study design: mice were randomized into five groups (n=10/group): vehicle control, isotype control, APRIL-treated, APRIL/BAFF-treated, or unvaccinated. Treatments were administered twice weekly for the duration of the study. Influenza vaccination was performed on day 28 and 49. On day 70, animals were challenged with a mouse adapted Cal09 influenza strain and monitored daily for clinical signs and survival until day 91. FIG. 4B depicts Kaplan-Meier survival curves for each group. **p=<0.01, log-rank (Mantel-Cox); *p=<0.05, log-rank (Mantel-Cox).

FIGS. 5A-5B depict serological and cellular responses to KLH vaccination. FIG. 5A depicts IgM response over time. FIG. 5B depicts IgG response over time. Data points represent mean±SEM for each group; comparison versus control (or isotype) at each time point is indicated (*p<0.05, **p<0.01, ***p<0.001). The dotted lines represent the lower limit of quantification.

### DETAILED DESCRIPTION

Disclosed herein are methods of treating IgA nephropathy or autoimmune diseases associated with APRIL (e.g., rheumatoid arthritis, systemic lupus erythematosus, a linear IgA bullous disease, IgA-mediated epidermolysis bullosa acquisita, or Sjögren's disease) in a subject who needs treatment for IgA nephropathy or an autoimmune disease and has a risk of an infection during the treatment by selectively administering the subject an anti-APRIL antibody instead of an APRIL/BAFF inhibitor. The present disclosure demonstrates that while both APRIL and APRIL/BAFF inhibitors reduce serum IgA, dual APRIL/BAFF inhibition leads to significant ablation of most B cell subsets and antibody-secreting cells, whereas APRIL-only inhibition unexpectedly does not substantially alter splenic B cell populations. Broad ablation of B cells has implications for immune homeostasis and B cell responses to pathogens during infection.

### DEFINITIONS

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods disclosed herein encompass polypeptides and nucleic acids having the sequences specified, or sequences substantially identical or similar thereto, e.g., sequences at least 85%, 90%, 95% identical or higher to the sequence specified.

In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical aspect, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some aspects, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In certain aspects, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and gapped BLAST programs, the default parameters of the respective programs (e.g., XBLAST and NBLAST) can be used. See www.ncbi.nlm.nih.gov.

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “treat,” e.g., IgA nephropathy or autoimmune diseases associated with APRIL (e.g., rheumatoid arthritis, systemic lupus erythematosus, a linear IgA bullous disease, IgA-mediated epidermolysis bullosa acquisita, or Sjögren's disease), means that a subject (e.g., a human) who has a disorder, e.g., IgA nephropathy or autoimmune diseases associated with APRIL (e.g., rheumatoid arthritis, systemic lupus erythematosus, a linear IgA bullous disease, IgA-mediated epidermolysis bullosa acquisita, or Sjögren's disease), and/or experiences a symptom of a disorder, e.g., IgA nephropathy or autoimmune diseases associated with APRIL (e.g., rheumatoid arthritis, systemic lupus erythematosus, a linear IgA bullous disease, IgA-mediated epidermolysis bullosa acquisita, or Sjögren's disease), will, in an aspect, suffer less a severe symptom and/or recover faster when an antibody is administered than if the antibody were never administered. Assays, urine tests, blood tests, iothalamate clearance tests, or kidney imaging (e.g., ultrasound, X-rays, or cystoscopy), for example, can be used to monitor treatment in a patient, or to detect the presence, e.g., decreased presence (or absence), of a symptom of IgA nephropathy, after treatment of IgA nephropathy in the subject. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., IgA nephropathy. In some aspects, treatment is of a subject who does not exhibit certain signs of a disorder, e.g., IgA nephropathy, and/or of a subject who exhibits only early signs of a disorder. In some aspects, treatment is of a subject who exhibits one or more established signs of a disorder, e.g., IgA nephropathy. In some aspects, treatment is of a subject diagnosed as suffering from a disorder, e.g., IgA nephropathy.

As used herein, the term “prevent,” a disorder, e.g., IgA nephropathy, means that a subject (e.g., a human) is less likely to have the disorder, e.g., IgA nephropathy, if the subject receives the antibody.

As used herein, the term “subject” is intended to include human and non-human animals. In some aspects, the subject is a human subject, e.g., a human patient having a disorder described herein (e.g., IgA nephropathy), or at risk of having a disorder described herein (e.g., IgA nephropathy). The term “non-human animals” includes mammals and non-mammals, such as non-human primates. The methods and compositions described herein are suitable for treating human patients having a disorder described herein (e.g., IgA nephropathy). Patients having a disorder described herein (e.g., IgA nephropathy) include those who have developed a disorder described herein (e.g., IgA nephropathy) but are (at least temporarily) asymptomatic, patients who have exhibited a symptom of a disorder described herein (e.g., IgA nephropathy), or patients having a disorder related to or associated with a disorder described herein (e.g., IgA nephropathy).

As used herein, the term “fusion protein” refers to a protein with two or more different protein domains integrated into one molecule. In some aspects, a fusion protein refers to a protein made from a fusion gene created by joining two or more genes. In some aspects, a fusion protein can be TACI-Fc fusion proteins povetacicept (POV) and atacicept (ATA), based on the sequences of povetacicept (ALPN-303) and atacicept (VT-001), respectively.

As used herein, the term “mild infection” refers to an infection that causes mild symptoms in an infected subject and/or an infection that does not pose a significant health threat to a subject. A mild infection may be resolved without medical intervention. As used herein, the term “moderate infection” refers to an infection that causes moderate symptoms in an infected subject and/or an infection that poses a moderate health threat to a subject. A moderate infection may be resolved with or without medical intervention. As used herein, the terms “severe and/or active infection,” “severe infection,” and “active infection” refer to an infection that causes severe symptoms in an infected subject and/or an infection that poses a significant health threat to a subject. A severe infection may be life-threatening. A severe infection is resolved with medical intervention. A subject having a moderate infection or a severe and/or active infection may exhibit an increased white blood cell count. Various aspects of the compositions and methods herein are described in further detail below. Additional definitions are set out throughout the specification.

### Methods of Treating IgA Nephropathy and Other Autoimmune Diseases in Subjects at Risk for an Infection During Treatment

The present disclosure provides methods of treating IgA nephropathy (IgAN) or autoimmune diseases associated with APRIL (e.g., rheumatoid arthritis, systemic lupus erythematosus, a linear IgA bullous disease, IgA-mediated epidermolysis bullosa acquisita, or Sjögren's disease) in a subject who needs treatment for IgA nephropathy or an autoimmune disease and has a risk of an infection during the treatment by selectively administering the subject an anti-APRIL antibody instead of an APRIL/BAFF inhibitor. In some aspects, the infection comprises a viral infection, a bacterial infection, a fungal infection, or a parasitic infection. In some aspects, the infection causes an infectious disease. In some aspects, the infectious disease comprises Coronavirus disease (COVID-19), influenza, pneumonia, or tuberculosis. In some aspects, administering the anti-APRIL antibody to the subject maintains the subject's B cell population(s).

### IgA Nephropathy

IgAN (also known as Berger's disease, Berger disease, Berger's syndrome, Berger syndrome, IgA nephritis, IgAN, or synpharyngitis glomerulonephritis) is the most prevalent, chronic glomerular disease worldwide. IgAN is an autoimmune disease caused by the deposition of IgA, typically in the form of immune complexes in the mesangium of the kidney. These IgAs are of the A1 subclass (IgAQ1 vs. IgA2), predominantly polymeric (with J chain-mediated linkages), and apparently differentially o-glycosylated in the hinge region that is intervening between CH1 and CH2 domains. In particular, these o-glycans are heterogeneously lacking β1,3 galactose linkages and, as such, are commonly referred to as galactose-deficient IgAQ1 (or gdIgA1). As the pathogenesis of this disease can involve a polygenic, multi-hit mechanism for inducing renal pathology and aberrant physiology, IgAQ1 may be viewed as the so-called auto-antigen representing this first critical “hit” in a multi-hit model for IgA nephropathy. A set of autoantibodies for this disease has likewise been defined and it relates to immunoglobulins (predominantly IgG) that specifically recognize this differentially glycosylated epitope and promote the formation of immune complexes (representing so-called “hit 2”). IgA itself is subject to aggregation due to misfolding, conformational changes, and potential changes in the N-glycosylation state of the CH2/CH3 glycans. And aberrantly glycosylated IgAQ1 levels have been associated with disease and clinical outcomes in IgAN.

### Other Autoimmune Diseases

Other autoimmune disease associated with APRIL include, e.g., Exemplary autoimmune disorders that can be treated or prevented by the antibody described herein include, but are not limited to, acute Disseminated Encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenia purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, Erythema nodosum, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's Granulomatosis), Graves' disease, Guillain-Barre syndrome, Hashimoto's encephalitis, Hashimoto's thyroiditis, hemolytic anemia, Henoch-Schonlein purpura, Herpes gestationis, hypogammaglobulinemia, idiopathic thrombocytopenia purpura (ITP), IgA nephropathy, IgG4-related sclerosing disease, IgA-mediated epidermolysis bullosa acquisita, immunoregulatory lipoproteins, inclusion body myositis, interstitial cystitis, juvenile arthritis, juvenile diabetes (type 1 diabetes), juvenile myositis, Kawasaki syndrome, Lambert-Eaton syndrome, Leukocytoclastic vasculitis, Lichen planus, Lichen sclerosus, Ligneous conjunctivitis, linear IgA disease (LAD), pupus (SLE), linear IgA bullous disease, Lyme disease, chronic, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, Pars planitis (peripheral uveitis), pemphigus, peripheral neuropathy, perivenous encephalomyelitis, pernicious anemia, POEMS syndrome, polyarteritis nodosa, Type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, raynauds phenomenon, reactive arthritis, reflex sympathetic dystrophy, reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's disease, sperm & testicular autoimmunity, stiff person syndrome, subacute bacterial endocarditis (SBE), Susac's syndrome, sympathetic ophthalmia, systemic lupus erythematosus, Takayasu's arteritis, temporal arteritis/Giant cell arteritis, thrombocytopenia purpura (TTP), Tolosa-Hunt syndrome, transverse myelitis, Type 1 diabetes, ulcerative colitis, undifferentiated connective tissue disease (UCTD), uveitis, vasculitis, vesiculobullous dermatosis, vitiligo, Wegener's granulomatosis (also known as Granulomatosis with Polyangiitis (GPA).

### Diagnosis of IgA Nephropathy

IgA nephropathy can be diagnosed by various tests, e.g., urine test, blood tests (e.g., to show increased blood levels of the waste product creatinine), iothalamate clearance test, kidney imaging (e.g., ultrasound, X-rays, or cystoscopy), kidney biopsy, or a combination thereof. For an adult patient with isolated hematuria, tests such as ultrasound of the kidney and cystoscopy are usually done first to pinpoint the source of the bleeding. These tests would rule out kidney stones and bladder cancer, two other common urological causes of hematuria. In children and younger adults, the history and association with respiratory infection can raise the suspicion of IgA nephropathy. A kidney biopsy is often necessary to confirm the diagnosis. The biopsy specimen shows proliferation of the mesangium, with IgA deposits on immunofluorescence and electron microscopy. However, patients with isolated microscopic hematuria (i.e., without associated proteinuria and with normal kidney function) are not usually biopsied since this is associated with an excellent prognosis. A urinalysis will show red blood cells, usually as red cell urinary casts. Proteinuria, usually less than 2 grams per day, also may be present. Other renal causes of isolated hematuria include, e.g., thin basement membrane disease and Alport syndrome, the latter being a hereditary disease associated with hearing impairment and eye problems. Other blood tests done to aid in the diagnosis include CRP or ESR, complement levels, ANA, and LDH. Protein electrophoresis and immunoglobulin levels can show increased IgA in 50% of all patients.

### APRIL as a Target

The identification and validation of select immune-based targets proposed to play a critical role in disease pathogenesis, namely the production of IgA and subsequent production of autoreactive antibodies to this target, represent a logical therapeutic strategy for treatment. APRIL (TNFSF13) represents particular area of focus for this reason. Additional rationale for targeting APRIL include emerging genetic data based on multiple, comprehensive genome wide association (GWAS) studies along with IgA related genetic disorders e.g., IgA hypogammaglobulinemia related common variable immunoglobulin deficiency (CVID) whose locus maps to defects in TNFRSF13B (TACI) with direct implications of the role of APRIL-TACI interactions in regulating IgA synthesis. The anti-APRIL antibodies described herein can be used to treat IgA nephropathy and other autoimmune disease associated with APRIL.

### BAFF as a Target

The identification and validation of select immune-based targets proposed to play a critical role in disease pathogenesis, namely the production of IgA and subsequent production of autoreactive antibodies to this target, represent a logical therapeutic strategy for treatment. BAFF represents a particular area of focus for this reason. Elevated circulating levels of Gd-IgAQ1 that trigger autoantibody production are associated with IgAN, resulting in the formation and deposition of immune complexes in the mesangium, leading to inflammation and kidney injury. BAFF is a key mediator of the production of Gd-IgAQ1 and its corresponding autoantibodies and plays an essential role in the survival and maintenance of B cells and humoral immunity. Elevated BAFF serum levels are also observed in patients with IgAN and correlate with disease severity. The BAFF inhibitors described herein can be used to treat IgA nephropathy and other autoimmune disease associated with BAFF.

### Anti-APRIL and Anti-BAFF Therapies

Disclosed herein are use of anti-APRIL therapies (e.g., an anti-APRIL antibody, an APRIL inhibitor, or an APRIL/BAFF inhibitor, described herein) and anti-BAFF therapies (e.g., a BAFF inhibitor or an APRIL/BAFF inhibitor, described herein), for treating IgA nephropathy or autoimmune diseases associated with APRIL (e.g., rheumatoid arthritis, systemic lupus erythematosus, a linear IgA bullous disease, IgA-mediated epidermolysis bullosa acquisita, or Sjögren's disease).

APRIL (A PRoliferation Inducing Ligand), also known as CD256, TNF-αnd APOL-related Leukocyte Expressed Ligand 2 (TALL-2), or TNF-related Death Ligand 1 (TRDL-1), is a TNF family cytokine encoded by the Tumor Necrosis Factor Ligand Superfamily Member 13 (TNFSF13) gene (also known as APRIL, TALL2, or ZTNF2). APRIL plays a role in a number of biological processes such as signal transduction, regulation of cell proliferation, and IgA class switching (Hahne et al. (1998) J. Exp. Med. 188:1185-1190 (1998); Castigli et al. Proc. Natl. Acad. Sci. U.S.A. 101:3903-3908 (2004)).

APRIL is both functionally and structurally related to BAFF (B Cell Activating Factor F13B) also known as BLyS (B lymphocyte stimulator). Both cytokines are involved in regulating key aspects of innate and adaptive immune functions. Exemplary amino acid and nucleotide sequences of human APRIL are described, e.g., in Hahne et al. J. Exp. Med. 188:1185-1190 (1998); Shu et al. J. Leukoc. Biol. 65:680-683 (1999); Kelly et al. Cancer Res. 60:1021-1027 (2000); and Pradet-Balade et al. EMBO J. 21:5711-5720 (2002).

The amino acid sequence of human APRIL (isoform alpha, also referred to as the “canonical” sequence (SEQ ID NO: 11)) is provided as follows.

>huAPRIL
MPASSPFLLAPKGPPGNMGGPVREPALSVALWLSWGAALGAVACAMALLT
QQTELQSLRREVSRLQGTGGPSQNGEGYPWQSLPEQSSDALEAWENGERS
RKRRAVLTQKQKKQHSVLHLVPINATSKDDSDVTEVMWQPALRRGRGLQA
QGYGVRIQDAGVYLLYSQVLFQDVTFTMGQVVSREGQGRQETLFRCIRSM
PSHPDRAYNSCYSAGVFHLHQGDILSVIIPRARAKLNLSPHGTFLGFVKL

### Molecules Useful for the Present Methods

As used herein, the term “antibody” refers to a protein, e.g., an immunoglobulin chain or a fragment thereof, comprising at least one immunoglobulin variable domain sequence. The term “antibody” includes, for example, full-length, mature antibodies and antigen-binding fragments of an antibody. For example, an antibody can include a heavy (H) chain variable domain sequence (abbreviated herein as VH), and a light (L) chain variable domain sequence (abbreviated herein as VL). In another example, an antibody includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments include: (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibody can be a single chain antibody. A single-chain antibody (scFv) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

The antibodies disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to some aspects, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also contemplated.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). In some aspects, the following definitions are used: AbM definition of CDR1 of the heavy chain variable domain and Kabat definitions for the other CDRs. In some aspects, Kabat definitions are used for all CDRs. In addition, aspects described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, and FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The terms “compete” or “cross-compete” are used interchangeably herein to refer to the ability of an antibody to interfere with binding of an anti-APRIL antibody, e.g., an anti-APRIL antibody provided herein, to a target, e.g., APRIL. The interference with binding can be direct or indirect (e.g., through an allosteric modulation of the antibody or the target). The extent to which an antibody is able to interfere with the binding of another antibody to the target, and therefore whether it can be said to compete, can be determined using a competition binding assay, for example, a FACS assay, an ELISA or BIACORE assay. In some aspects, a competition binding assay is a quantitative competition assay. In some aspects, a first anti-APRIL antibody is said to compete for binding to the target with a second anti-APRIL antibody when the binding of the first antibody to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibodies of single molecular composition. A monoclonal antibody composition displays a single binding specificity and affinity for a particular epitope. A monoclonal antibody can be made by hybridoma technology or by methods that do not use hybridoma technology (e.g., recombinant methods),

An “effectively human” protein is a protein that does not evoke a neutralizing antibody response, e.g., the human anti-murine antibody (HAMA) response. HAMA can be problematic in a number of circumstances, e.g., if the antibody is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody can be a polyclonal or a monoclonal antibody. In some aspects, the antibody can be recombinantly produced, e.g., produced by any suitable phage display or combinatorial methods.

In some aspects, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. In some aspects, the non-human antibody is a rodent (mouse or rat antibody). Methods of producing rodent antibodies are known in the art.

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In some aspects, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some aspects, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

In some aspects, the antibody has a heavy chain constant region chosen from, e.g., the heavy chain constant regions of IgG1, IgG2 (e.g., IgG2a), IgG3, IgG4, IgM, IgA1, IgA2, IgD, and IgE; particularly, chosen from, e.g., the (e.g., human) heavy chain constant regions of IgG1, IgG2, IgG3, and IgG4. In another aspect, the antibody has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some aspects, the antibody has effector function and can fix complement. In another aspect, the antibody does not recruit effector cells or fix complement. In certain aspects, the antibody has reduced or no ability to bind an Fc receptor. For example, it may be an isotype or subtype, fragment or other mutant, which does not support binding to an Fc receptor, e.g., it has a mutagenized or deleted Fc receptor binding region.

In some aspects, this disclosure provides a method of making an antibody disclosed herein. The method includes: providing an antigen, e.g., APRIL or a fragment thereof; obtaining an antibody that specifically binds to the antigen; evaluating efficacy of the antibody in modulating activity of the antigen and/or organism expressing the antigen, e.g., APRIL. The method can further include administering the antibody, including a derivative thereof (e.g., a humanized antibody) to a subject, e.g., a human.

Amino acid sequences of exemplary anti-APRIL antibodies and/or exemplary APRIL inhibitors useful as an anti-APRIL therapy in the methods of the present disclosure are described in Tables 1 and 2.

Amino acid sequences of additional exemplary antibodies and exemplary fusion proteins are described in Table 3.

TABLE 1
The amino acid sequences of the heavy chain variable region (VH) and light chain
variable region (VL) of an exemplary anti-APRIL antibody and/or an exemplary APRIL
inhibitor (e.g., Sibeprenlimab) are provided as follows. CDRs are defined according to
the Chothia system are underlined.

| | | SEQ ID | | | SEQ |
|---|---|---|---|---|---|
| Chain | Amino Acid Sequence | NO | Chothia CDR | | ID NO |
| VH | QVQLVQSGAEVKKPGASVKVSCKASGYTFTDYTIHWV | 1 | HCDR | GYTFTDY | 3 |
| | RQATGQGLEWMGWIYPLRGSINYAQKFQGRVTMTAD | | 1 | | |
| | KSISTVYMELSSLRSEDTAVYFCARHGAYYSNAFDYW | | HCDR | YPLRGS | 4 |
| | GQGTLVTVSS | | 2 | | |
| | | | HCDR | HGAYYSNAFD | 5 |
| | | | 3 | Y | |
| VL | EIVMTQSPATLSVSPGERATLSCRASESVDNDGIRFLH | 2 | LCDR1 | RASESVDNDG | 6 |
| | WYQQKPGQAPRLLIYRASTRATGIPARFSGSGSRTEFTL | | | IRFLH | |
| | TISSLQSEDFAVYYCQQSNKDPYTFGGGTKVEIK | | LCDR2 | RASTRAT | 7 |
| | | | LCDR3 | QQSNKDPYT | 8 |

TABLE 2
The amino acid sequences of the heavy chain (HC) and light chain (LC) of an exemplary anti-
APRIL antibody and/or an exemplary APRIL inhibitor (e.g., Sibeprenlimab) are provided
below.

| Feature | Sequence | SEQ ID NO: |
|---|---|---|
| Heavy | QVQLVQSGAEVKKPGASVKVSCKASGYTFTDYTIHWVRQATGQGLEWMGWIYPLRGSINY | 9 |
| Chain | AQKFQGRVTMTADKSISTVYMELSSLRSEDTAVYFCARHGAYYSNAFDYWGQGTLVTVSSA | |
| | STKGPSVFPLAPCSRSTSESTAALGCLVKDYFPEPVTVSWNSGALTSGVHTFPAVLQSSGLYS | |
| | LSSVVTVPSSNFGTQTYTCNVDHKPSNTKVDKTVERKCCVECPPCPAPPVAGPSVFLFPPKPK | |
| | DTLMISRTPEVTCVVVDVSHEDPEVQFNWYVDGVEVHNAKTKPREEQFNSTFRVVSVLTVV | |
| | HQDWLNGKEYKCKVSNKGLPAPIEKTISKTKGQPREPQVYTLPPSREEMTKNQVSLTCLVKG | |
| | FYPSDIAVEWESNGQPENNYKTTPPMLDSDGSFFLYSKLTVDKSRWQQGNVFSCSVMHEAL | |
| | HNHYTQKSLSLSPGK | |
| Light | EIVMTQSPATLSVSPGERATLSCRASESVDNDGIRFLHWYQQKPGQAPRLLIYRASTRATGIPA | 10 |
| Chain | RFSGSGSRTEFTLTISSLQSEDFAVYYCQQSNKDPYTFGGGTKVEIKRTVAAPSVFIFPPSDEQL | |
| | KSGTASVVCLLNNFYPREAKVQWKVDNALQSGNSQESVTEQDSKDSTYSLSSTLTLSKADYE | |
| | KHKVYACEVTHQGLSSPVTKSFNRGEC | |

TABLE 3
The amino acid sequences of additional exemplary antibodies and exemplary fusion proteins are
provided below.

| Antibody | Feature | Sequence | SEQ ID NO: |
|---|---|---|---|
| 4540 anti- | Heavy | QVQLQQSGPELVKPGASVKISCKASGYTFADYYINWVKQRPGQGLEWIGWIFP | 12 |
| APRIL mAb | chain | GSGSTYYNEKFKGKATLTVDKSSSTAYMLLSSLTSEDSAVYFCARGDSGRAMD | |
| | | YWGQGTSVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDK | |
| | | KVEPKSCDKTHTCPPCPAPEVDGGPSVFLFPPKPKDTLMISRTPEVTCVVVGVSH | |
| | | EDPEVKFNWYVDGVEVHNAKTKPREEQYNSTYRVVSVLTVLHQDWLNGKEY | |
| | | KCKVSNKALPAPIEKTISKAKGQPREPQVYTLPPSRDELTKNQVSLTCLVKGFYP | |
| | | SDIAVEWESNGQPENNYKTTPPVLDSDGSFFLYSKLTVDKSRWQQGNVFSCSV | |
| | | MHEALHNHYTQKSLSLSPGK* | |
| 4540 anti- | Light | DIQMTQSPSSLSASLGGKVTITCKASQDINKYIAWYQHKPGKGPRLLIHYTSTLQ | 13 |
| APRIL mAb | chain | SGIPSRFSGSGSGRDYSFSISNLEPEDNATYYCLQYDNLLTFGAGTKLELKRTVA | |
| | | APSVFIFPPSDEQLKSGTASVVCLLNNFYPREAKVQWKVDNALQSGNSQESVTE | |
| | | QDSKDSTYSLSSTLTLSKADYEKHKVYACEVTHQGLSSPVTKSFNRGEC* | |
| POV001; | — | SLSCRKEQGEYYDHLLRDCISCASICGQHPKQCADFCENKLRSGSGGGGSEPKS | 14 |
| Based on | | SDKTHTCPPCPAPEAEGAPSVFLFPPKPKDTLMISRTPEVTCVVVDVSHEDPEVK | |
| Povetacicept | | FNWYVDGVEVHNAKTKPREEQYNSTYRVVSVLTVLHQDWLNGKEYKCKVSN | |
| | | KALPAPIEKTISKAKGQPREPQVYTLPPSRDELTKNQVSLTCLVKGFYPSDIAVE | |
| | | WESNGQPENNYKTTPPVLDSDGSFFLYSKLTVDKSRWQQGNVFSCSVMHEAL | |
| | | HNHYTQKSLSLSPG* | |
| ATA001; | — | AMRSCPEEQYWDPLLGTCMSCKTICNHQSQRTCAAFCRSLSCRKEQGKFYDHL | 15 |
| Based on | | LRDCISCASICGQHPKQCAYFCENKLRSEPKSSDKTHTCPPCPAPEAEGAPSVFL | |
| Atacicept | | FPPKPKDTLMISRTPEVTCVVVDVSHEDPEVKFNWYVDGVEVHNAKTKPREEQ | |
| | | YNSTYRVVSVLTVLHQDWLNGKEYKCKVSNKALPSSIEKTISKAKGQPREPQV | |
| | | YTLPPSRDELTKNQVSLTCLVKGFYPSDIAVEWESNGQPENNYKTTPPVLDSDG | |
| | | SFFLYSKLTVDKSRWQQGNVFSCSVMHEALHNHYTQKSLSLSPGK* | |
| MOTA; | VH | QVTLRESGPALVKPTQTLTLTCTFSGFSLSTAGMSVGWIRQPPGKALEWLADIW | 16 |
| Based on | | WDDKKHYNPSLKDRLTISKDTSKNQVVLKVTNMDPADTATYYCARDMIFNFY | |
| Motavizumab | | FDVWGQGTTVTVSSASTKGPSVFPLAPSSKSTSGGTAALGCLVKDYFPEPVTVS | |
| | | WNSGALTSGVHTFPAVLQSSGLYSLSSVVTVPSSSLGTQTYICNVNHKPSNTKV | |
| | | DKRVEPKSCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQDWLNGK | |
| | | EYKCKVSNKALPAPIEKTISKAKGQPREPQVYTLPPSREEMTKNQVSLTCLVKG | |
| | | FYPSDIAVEWESNGQPENNYKTTPPVLDSDGSFFLYSKLTVDKSRWQQGNVFSC | |
| | | SVMHEALHNHYTQKSLSLSPGK | |
| MOTA; | VL | DIQMTQSPSTLSASVGDRVTITCSASSRVGYMHWYQQKPGKAPKLLIYDTSKLA | 17 |
| Based on | | SGVPSRFSGSGSGTEFTLTISSLQPDDFATYYCFQGSGYPFTFGGGTKVEIKRTVA | |
| Motavizumab | | APSVFIFPPSDEQLKSGTASVVCLLNNFYPREAKVQWKVDNALQSGNSQESVTE | |
| | | QDSKDSTYSLSSTLTLSKADYEKHKVYACEVTHQGLSSPVTKSFNRGEC | |

In some aspects, the antibody useful for the present methods comprises an antibody described herein, e.g., an antibody described in Tables 1-3 (corresponding to SEQ ID NOs: 1-17). In some aspects, the antibody comprises one, two, or three CDRs of the VH region and/or the VL region of an antibody described herein, e.g., in Table 1 (e.g. Sibeprenlimab). In some aspects, the antibody comprises a heavy chain variable region having the amino acid sequence as set forth in SEQ ID NO: 1 and/or a light chain variable region having the amino acid sequence as set forth in SEQ ID NO: 2. In some aspects, the antibody comprises a heavy chain having the amino acid sequence as set forth in SEQ ID NO: 12 and/or a light chain having the amino acid sequence as set forth in SEQ ID NO: 13.

In some aspects, the antibody further comprises a heavy chain constant region. In some aspects, the heavy chain constant region is an IgG1 constant region, or a functional portion thereof. In another aspect, the heavy chain constant region is an IgG2 constant region, or a functional portion thereof. In some aspects, the antibody further comprises a light chain constant region. In some aspects, the antibody further comprises a heavy chain constant region and a light chain constant region.

In some aspects, the antibody useful for the present methods comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising an amino acid sequence that differs by no more than 1, 2, or 3 amino acid residues from, or has at least 85, 90, 95, 99 or 100% homology with, the amino acid sequence of SEQ ID NO: 3; an HCDR2 comprising an amino acid sequence that differs by no more than 1, 2, or 3 amino acid residues from, or has at least 85, 90, 95, 99 or 100% homology with, the amino acid sequence of SEQ ID NO: 4; or an HCDR3 comprising an amino acid sequence that differs by no more than 1, 2, or 3 amino acid residues from, or has at least 85, 90, 95, 99 or 100% homology with, the amino acid sequence of SEQ ID NO: 5, or
(ii) a light chain variable region (VL), wherein the light chain variable region comprises three light chain complementarity determining regions (LCDR1, LCDR2, and LCDR3), wherein the light chain variable region comprises: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 6; an LCDR2 comprising an amino acid sequence that differs by no more than 1, 2, or 3 amino acid residues from, or has at least 85, 90, 95, 99 or 100% homology with, the amino acid sequence of SEQ ID NO: 7; or an LCDR3 comprising an amino acid sequence that differs by no more than 1, 2, or 3 amino acid residues from, or has at least 85, 90, 95, 99 or 100% homology with, the amino acid sequence of SEQ ID NO: 8.

In some aspects, the antibody useful for the present methods comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 3; an HCDR2 comprising the amino acid sequence of SEQ ID NO: 4; and an HCDR3 comprising the amino acid sequence of SEQ ID NO: 5, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 6; an LCDR2 comprising the amino acid sequence of SEQ ID NO: 7; or an LCDR3 comprising the amino acid sequence of SEQ ID NO: 8.

In some aspects, the antibody comprises a VH comprising an amino acid sequence having at least 85, 90, 95, 99 or 100% sequence identity to SEQ ID NO: 1. In some aspects, the antibody comprises a VL comprising an amino acid sequence having at least 85, 90, 95, 99 or 100% sequence identity to SEQ ID NO: 2. In some aspects, the antibody comprises a VH comprising the amino acid sequence as set forth in SEQ ID NO: 1. In some aspects, the antibody comprises a VL comprising the amino acid sequence as set forth in SEQ ID NO: 2.

In some aspects, the antibody useful for the present methods comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 3; an HCDR2 comprising the amino acid sequence of SEQ ID NO: 4; and an HCDR3 comprising the amino acid sequence of SEQ ID NO: 5, and wherein the VH comprises an amino acid sequence having at least 85, 90, 95, 99 or 100% sequence identity to SEQ ID NO: 1; and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 6; an LCDR2 comprising the amino acid sequence of SEQ ID NO: 7; or an LCDR3 comprising the amino acid sequence of SEQ ID NO: 8 and wherein the VL comprises an amino acid sequence having at least 85, 90, 95, 99 or 100% sequence identity to SEQ ID NO: 2.

In some aspects, the antibody comprises a heavy chain comprising an amino acid sequence having at least 85, 90, 95, 99 or 100% sequence identity to SEQ ID NO: 9. In some aspects, the antibody comprises a VL comprising an amino acid sequence having at least 85, 90, 95, 99 or 100% sequence identity to SEQ ID NO: 10. In some aspects, the antibody comprises a VH comprising the amino acid sequence as set forth in SEQ ID NO: 9 and a VL comprising the amino acid sequence as set forth in SEQ ID NO: 10. In some aspects, the antibody comprises a heavy chain comprising an amino acid sequence having at least 85, 90, 95, 99 or 100% sequence identity to SEQ ID NO: 12. In some aspects, the antibody comprises a light chain comprising an amino acid sequence having at least 85, 90, 95, 99 or 100% sequence identity to SEQ ID NO: 13.

In some aspects, the antibody useful for the present methods has one or more (e.g., 2, 3, 4, 5, or all) of the following properties: (a) is a humanized antibody; (b) binds to human APRIL at an EC₅₀ of 60 μM or less, as determined by ELISA; (c) inhibits binding of human APRIL to TACI, e.g., in vitro, at an IC₅₀ of 0.5 nM or less; (d) inhibits binding of human APRIL to BCMI, e.g., in vitro, at an IC₅₀ of 0.6 nM or less; (e) is an IgG2K; or (f) has an Fc region engineered to reduce complement activation. In some aspects, the antibody comprises one or more (e.g., 2, 3, 4, 5, or all) CDRs, one or both of heavy chain variable region or light chain variable regions, or one or both of heavy chain or light chain, of sibeprenlimab.

In some aspects, the antibody binds, or substantially binds, to an epitope that comprises one or more APRIL residues from the C-D loop (e.g., the loop connecting β-sheets C and D), the G-H loop (e.g., the loop connecting B-sheets G and H), or both. In some aspects, the antibody binds, or substantially binds, to one or more (e.g., 2, 3, 4, 5, 6, 7, 8, 9, or all) residues of human APRIL from positions 105-114 and/or one or more (e.g., 2, 3, 4, 5, 6, 7, 8, 9, or all) residues of mouse APRIL from positions 96-105. In some aspects, the epitope is a conformational epitope.

In some aspects, the antibody does not bind, or does not substantially bind, to one, two or all of Asp129, Arg233, or His203 of human APRIL.

In some aspects, binding of the antibody to APRIL (e.g., human APRIL) inhibits, or substantially inhibits, the binding of the CRD2 domain of TACI (e.g., human TACI) to APRIL (e.g., human APRIL). In another aspect, binding of the antibody to human APRIL, inhibits, or substantially inhibits, the binding of human TACI, to one or more, e.g., 2, 3, 4, 5, 6, 7, 8, 9, 10, 11, 12, 13, 14, 15, 16, 17, 18, 19, 20, 21, 22, 23, 24, 25, or all of the APRIL residues.

In some aspects, the antibody useful for the present disclosure comprises Sibeprenlimab. In some aspects, the antibody useful for the present disclosure comprises Zigakibart/BION-1301. See WO 2010/100056, published Sep. 10, 2010; WO2015/034364, published Mar. 12, 2015; WO2016/110587, published Jul. 14, 2016; WO2021/243298, published Dec. 2, 2021; and WO2024/105549, published May 23, 2024, incorporated herein by reference in their entireties.

In some aspects, an APRIL/BAFF inhibitor useful for the present disclosure comprises a fusion protein. In some aspects, an APRIL/BAFF inhibitor useful for the present disclosure comprises atacicept, povetacicept, or telitacicept. In some aspects, an APRIL/BAFF inhibitor useful for the present disclosure comprises atacicept. In some aspects, an APRIL/BAFF inhibitor useful for the present disclosure comprises povetacicept. In some aspects, an APRIL/BAFF inhibitor useful for the present disclosure comprises telitacicept. In some aspects, an APRIL/BAFF inhibitor useful for the present disclosure comprises SEQ ID Nos. 14 or 15.

In some aspects, an anti-APRIL therapy that is an APRIL/BAFF inhibitor comprises co-administering an APRIL inhibitor and a BAFF inhibitor. In some aspects an APRIL inhibitor useful for the present disclosure comprises Sibeprenlimab. In some aspects, an APRIL inhibitor useful for the present disclosure comprises Zigakibart/BION-1301. See WO 2010/100056, published Sep. 10, 2010; WO2015/034364, published Mar. 12, 2015; WO2016/110587, published Jul. 14, 2016; WO2021/243298, published Dec. 2, 2021; and WO2024/105549, published May 23, 2024, incorporated herein by reference in their entireties. In some aspects, an APRIL inhibitor useful for the present disclosure comprises a protein disclosed in WO2024/123675, incorporated herein by reference in its entirety.

In some aspects, a BAFF inhibitor useful for the present disclosure comprises belimumab, tabalumab, AMG570, blisibimod, ianalumab, or Sandy-2.

### B Cells

B cells (also called B lymphocytes) are a type of white blood cell (also called lymphocytes) that produce antibodies and thus comprise part of the immune system. There are four main types of B cells: transitional (T1/T2 B cells), mature naïve B cells, plasma B cells, and memory B cells. Anaya J M, Shoenfeld Y, Rojas-Villarraga A, et al., editors. Bogota (Colombia): El Rosario University Press; 2013 Jul. 18.

T1/T2 B cells are transitional B cells that develop from immature B cells. T1 B cells are found in the bone marrow, blood, and spleen, while T2 B cells are restricted to the spleen. T1/T2 B cells cannot perform any actions to help fend off harmful pathogens; however, while they travel between bone marrow and secondary lymphoid tissues, they are subjected to checks to confirm they will not produce autoantibodies or antibodies that attack the host. Depending on signals received through the B-cell receptor (BCR) and other receptors, T2 B cells differentiate in mature naïve B cells.

There are three main subsets of mature naïve B cells: follicular B cells, marginal zone B cells, and B-1 B cells, each of which can differentiate into plasma cells when they interact with an antigen. Follicular (FO) B cells (also called B2 B cells) are the largest subpopulation of B cells and participate in the vast majority of responses to exogenous antigens. Their differentiation is influenced by a variety of factors including chemokines, BCR signaling, and antigens. They participate in T-cell dependent immune responses by using the BCR to engulf antigens, process them, and present them to the antigen-specific T Cells. Marginal Zone (MZ) B cells are located as a sentinel in the marginal zones of the spleen, which correspond to the interphase between the circulation and the splenic lymphoid tissue. MZ B cells express high levels of toll-like receptors (TLRs), which allow them to act as bridge between the innate and adaptive immune responses. MZ B cells can rapidly respond to an antigen-specific stimulus by using both T-cell dependent and independent mechanisms. B-1 B cells are the first B cells to form in the fetal liver and respond rapidly to antigen-specific stimuli in a T-cell independent manner. B-1 B cells are the main source of circulating antibodies.

Each of the three main subsets of mature naïve B cells, i.e., follicular B cells, marginal zone B cells, and B-1 B cells, can differentiate into short-lived, antibody-producing cells called plasmablasts that maintain the ability to divide and proliferate. Plasmablasts mature into plasma cells, which lose their ability to divide, but secrete large quantities of antibodies in response to specific antigens. Memory B cells are another type of matured B cell that remember particular antigens, and switched memory B cells are memory B cells that have undergone isotype switching. Regulatory B cells (Bregs) restrict the excessive inflammatory responses that are produced during autoimmune diseases or that can be caused by unresolved infections.

### Composition for Use in Treating an IgA Nephropathy or Autoimmune Disease

In some aspects a subject who needs treatment for IgA nephropathy or an autoimmune disease and has a risk of infection can be treated for IgA nephropathy or an autoimmune disease by selectively administering composition comprising an anti-APRIL antibody to the subject.

The present disclosure provides a composition comprising an anti-APRIL therapy suitable for use in treating an IgA nephropathy or autoimmune disease in a subject in need thereof, wherein the use comprises (a) identifying a subject who needs a treatment for an IgA nephropathy or autoimmune disease and has a risk of an infection during the treatment, (b) selecting, among anti-APRIL therapies comprising an anti-APRIL antibody and an APRIL/BAFF inhibitor, an anti-APRIL antibody as the anti-APRIL therapy suitable for the subject, and (c) administering the anti-APRIL antibody to the subject. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

### Method of Providing an Anti-APRIL Therapy for Treating an IgA Nephropathy or Autoimmune Disease

In some aspects a subject who needs treatment for IgA nephropathy or an autoimmune disease and has a risk of infection can be treated for IgA nephropathy or the autoimmune disease by selectively administering an anti-APRIL antibody to the subject.

In some aspects, the present disclosure provides a method of providing an anti-APRIL therapy for treating an IgA nephropathy or autoimmune disease in a subject in need thereof (a) identifying a subject who needs a treatment for an IgA nephropathy or autoimmune disease and has a risk of an infection during the treatment, (b) selecting, among anti-APRIL therapies comprising an anti-APRIL antibody and an APRIL/BAFF inhibitor, an anti-APRIL antibody as an anti-APRIL therapy suitable for the subject, and (c) administering the anti-APRIL antibody to the subject. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

### Method of Identifying a Subject Suitable for the Treatment of an Anti-APRIL Therapy & Selecting an Anti-APRIL Therapy

In some aspects, a subject suitable for treatment of an anti-APRIL therapy can be identified by assessing the subject's risk of infection during treatment and selecting an anti-APRIL antibody if the subject is identified as having a risk of infection during treatment.

In some aspects, the present disclosure provides a method of identifying a subject suitable for the treatment of an anti-APRIL therapy comprising (a) assessing the subject's risk of infection during the treatment and (b) selecting an anti-APRIL antibody as a suitable treatment if the subject has a risk of infection during the treatment. In some aspects, the method further comprises administering to the subject an anti-APRIL antibody. In some aspects, the anti-APRIL therapy comprises an anti-APRIL antibody therapy or an APRIL/BAFF inhibitor therapy. In some aspects, the subject is in need of treatment for an IgA nephropathy or autoimmune disease. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

### Method of Reducing or Preventing a Risk of Infection in a Subject in Need of an Anti-APRIL Therapy

In some aspects, the risk of a subject developing an infection can be reduced or prevented by administering to the subject an anti-APRIL antibody or APRIL inhibitor.

In some aspects, the present disclosure provides a method of reducing or preventing a risk of infection in a subject in need of an anti-APRIL therapy comprising administering to the subject an anti-APRIL antibody. In some aspects, the anti-APRIL therapy comprises an anti-APRIL antibody therapy or an APRIL/BAFF inhibitor therapy. In some aspects, the subject is in need of treatment for an IgA nephropathy or autoimmune disease. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

### Patient Population That Can Be Treated by Anti-APRIL Antibody

In some aspects, the patient population that can be treated for IgA nephropathy or an autoimmune disease with an anti-APRIL antibody or APRIL inhibitor comprises a patient population having an increased risk of infection or a patient population having developed an infection.

In some aspects, the present disclosure provides a method of treating an IgA nephropathy or autoimmune disease in a subject in need thereof comprising administering to the subject an anti-APRIL antibody, wherein the subject is identified as having a risk of infection during the treatment. In some aspects, the present disclosure provides a method of treating an IgA nephropathy or autoimmune disease in a subject in need thereof comprising administering to the subject an anti-APRIL antibody after the subject has developed an infection.

### Therapy Switching

In some aspects, an APRIL/BAFF inhibitor is administered to a subject in need of treatment for IgA nephropathy or an autoimmune disease before the subject is administered an anti-APRIL antibody or APRIL inhibitor.

In some aspects, the subject is administered an APRIL/BAFF inhibitor prior to the anti-APRIL antibody. In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the anti-APRIL antibody is administered subcutaneously. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the subject does not have a severe and/or active infection. In some aspects, the subject has a mild infection. In some aspects, the subject has a moderate infection. In some aspects, the anti-APRIL antibody or the APRIL inhibitor is administered without treatment interruption or discontinuation.

### Method of Maintaining B Cell Population

In some aspects, a B cell population in a subject in need of treatment for IgA nephropathy or an autoimmune disease is maintained by administering an anti-APRIL antibody or an APRIL inhibitor to the subject.

In some aspects, administering does not alter B cell population in the subject. In some aspects, administering preserves the B cell population in the subject. In some aspects, the B cell population is not altered in the spleen, bone marrow, and/or mesenteric lymph nodes. In some aspects, the B cell population is preserved in the spleen, bone marrow, and/or mesenteric lymph nodes. In some aspects, the B cell population is altered by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%. In some aspects, the B cell population is preserved by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, or about 90%. In some aspects, the present disclosure provides a method of maintaining B cell population during an anti-APRIL therapy in a subject in need of the therapy comprising administering to the subject an anti-APRIL antibody. In some aspects, the B cell population comprises total B cells, naïve B cells, T1/T2 B cells, follicular (FO) B cells, marginal zone (MZ) B cells, memory B cells, switched memory B cells, plasmablasts, B-1 cells, or regulatory B (Breg) cells.

### B Cell Depletion by APRIL/BAFF Inhibitors

In some aspects, treating a subject for IgA nephropathy or an autoimmune disease with an APRIL/BAFF inhibitor reduces the B cell population in the subject.

In some aspects, the present disclosure provides a method of treating an autoimmune disease in a subject in need thereof, comprising administering to the subject an APRIL/BAFF inhibitor, wherein the method reduces the B cell population. In some aspects, the B cell population comprises total B cells, naïve B cells, T1/T2 B cells, follicular (FO) B cells, marginal zone (MZ) B cells, plasma cells, memory B cells, switched memory B cells, plasmablasts, B-1 cells, or regulatory B (Breg) cells. In some aspects, the method reduces the population of T follicular helper (Tfh) cells. In some aspects, the APRIL/BAFF inhibitor comprises a fusion protein. In some aspects, the APRIL/BAFF inhibitor comprises, atacicept, povetacicept, or telitacicept. In some aspects, the APRIL/BAFF inhibitor comprises atacicept. In some aspects, the APRIL/BAFF inhibitor comprises povetacicept. In some aspects, the APRIL/BAFF inhibitor comprises telitacicept. In some aspects, the autoimmune disease comprises rheumatoid arthritis, systemic lupus erythematosus, a linear IgA bullous disease, IgA-mediated epidermolysis bullosa acquisita, or Sjögren's disease.

### ADDITIONAL ASPECTS

In some aspects, administering an anti-APRIL antibody to a subject does not alter the population of T follicular helper (Tfh) cells. In some aspects, administering an anti-APRIL antibody to a subject preserves the population of T follicular helper (Tfh) cells. In some aspects, the population of T follicular helper (Tfh) cells is altered by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%. In some aspects, the population of T follicular helper (Tfh) cells is preserved by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, or about 90%.

In some aspects, administering an anti-APRIL antibody to a subject does not alter the B cell population compared to an APRIL/BAFF inhibitor. In some aspects, administering an anti-APRIL antibody to a subject preserves the B cell population compared to an APRIL/BAFF inhibitor. In some aspects, the B cell population is not altered in the spleen, bone marrow, and/or mesenteric lymph nodes. In some aspects, the B cell population is preserved in the spleen, bone marrow, and/or mesenteric lymph nodes. In some aspects, the B cell population is altered by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%. In some aspects, the B cell population is preserved by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, or about 90%.

In some aspects, the B cell population comprises total B cells, naïve B cells, T1/T2 B cells, follicular (FO) B cells, marginal zone (MZ) B cells, memory B cells, switched memory B cells, plasmablasts, B-1 cells, or regulatory B (Breg) cells. In some aspects, administering an anti-APRIL antibody to a subject reduces IgA. In some aspects, administering an anti-APRIL antibody to a subject does not reduce IgM and/or IgG. In some aspects, IgM is reduced by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 20%, about 30%, about 40%, about 50%, about 55%, about 56%, about 57%, about 58%, or about 59%. In some aspects, IgG is reduced by only about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 16%, about 17%, about 18%, about 19%, about 20%, about 25%, about 30%, about 35%, about 40%, about 45%, or about 50%.

In some aspects, an anti-APRIL antibody useful for the compositions and methods described herein is administered to a subject at risk of an infection or a subject having an infection. In some aspects, the infection comprises a viral infection, a bacterial infection, a fungal infection, or a parasitic infection. Examples of viral infections include respiratory infections, such as the Common cold (usually caused by rhinovirus), the flu (influenza), COVID-19, respiratory syncytial virus (RSV), human metapneumovirus (hMPV), parainfluenza; digestive system infections, such as norovirus, rotavirus and astrovirus, and hepatitis viruses; viral hemorrhagic fevers, such as Ebola, hantavirus pulmonary syndrome, severe dengue, and yellow fever; sexually transmitted infections (STIs), such as human immunodeficiency virus (HIV), human papilloma virus (HPV)/genital warts, genital herpes (HSV), hepatitis B; exanthematous (rash-causing) infections, such as chickenpox, measles, rubella (German measles), roseola, fifth disease, and mpox; neurological infections, such as west nile virus, polio, and rabies; and congenital infections, such as cytomegalovirus (CMV), rubella, and zika virus. Examples of bacterial infections include Campylobacter and Salmonella infections, common causes of food poisoning; cellulitis, boils, impetigo, and skin infections; pneumococcal disease, including ear and sinus infections and some types of pneumonia; Lyme disease; bacterial vaginosis, an overgrowth of vaginal bacteria; Chlamydia and gonorrhea, sexually transmitted infections; strep throat; C. difficile, an intestinal infection, and E. coli, a common cause of urinary tract infection (UTI). Examples of fungal infections include superficial or mucocutaneous infections, such as ringworm (dermatophytosis), onychomycosis, candidiasis, and tinea versicolor/pityriasis versicolor; subcutaneous infections, such as sporotrichosis (rose gardener's disease), chromoblastomycosis, and eumycetoma; and deep infections, such as histoplasmosis, coccidioidomycosis (Valley fever), blastomycosis, aspergillosis, candidal urinary tract infection, invasive candidiasis, pneumocystis pneumonia (PJP), mucormycosis, and cryptococcosis. Examples of parasitic infections include infections caused by protozoa, helminths, and ectoparasites, such as malaria, toxoplasmosis, head lice, giardiasis, pinworms, trichomoniasis, cryptosporidiosis, cyclosporiasis, cysticercosis, strongyloidiasis, tapeworm infections, chagas disease, leishmaniasis, and schistosomiasis.

In some aspects, the infection causes an infectious disease. In some aspects, the infectious disease comprises nasopharyngitis, Coronavirus disease (COVID-19), influenza, sinusitis, pneumonia, or tuberculosis. In some aspects, the autoimmune disease comprises rheumatoid arthritis, systemic lupus erythematosus, a linear IgA bullous disease, IgA-mediated epidermolysis bullosa acquisita, or Sjögren's disease. In some aspects, the anti-APRIL therapy that is an APRIL/BAFF inhibitor comprises co-administering an APRIL inhibitor and a BAFF inhibitor. In some aspects, the APRIL inhibitor comprises zigakibart/BION-1301 or sibeprenlimab. In some aspects, the BAFF inhibitor comprises belimumab, tabalumab, AMG570, blisibimod, or ianalumab.

### Administration and Dosing

In some aspects, the anti-APRIL antibody for treating IgA nephropathy or an autoimmune disease is administered subcutaneously. In some aspects, the anti-APRIL antibody is administered intravenously (“IV”), i.e., via IV infusions. In some aspects, the anti-APRIL antibody is administered at a fixed dose between 10 mg and 1000 mg. In some aspects, the anti-APRIL antibody is administered at a fixed dose of 400 mg once every four weeks. In some aspects, the anti-APRIL antibody is administered at a fixed dose of 600 mg once every two weeks. In some aspects, the anti-APRIL antibody is administered at a weight-based dose between 2 and 50 mg/kg. In some aspects, the anti-APRIL antibody is administered at 2 mg/kg monthly via 12 monthly IV infusions. In some aspects, the anti-APRIL antibody is administered at 4 mg/kg monthly via 12 monthly IV infusions. In some aspects, the anti-APRIL antibody is administered at 8 mg/kg monthly via 12 monthly IV infusions. In some aspects, the anti-APRIL antibody is administered at 10 mg/kg. In some aspects, the anti-APRIL antibody is administered at 10 mg/kg twice weekly. In some aspects, the anti-APRIL antibody or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks.

### Pharmaceutical Composition

In some aspects, the present disclosure provides a pharmaceutical composition comprising: (a) an anti-APRIL antibody and (b) one or more ingredients comprising L-arginine, L-glutamic acid, L-histidine, L-histidine monohydrochloride, polysorbate 80, and/or sorbitol. In some aspects the present disclosure provides a pharmaceutical composition comprising: (a) an anti-APRIL antibody and (b) one or more ingredients comprising L-arginine, L-glutamic acid, L-histidine, polysorbate 80, and/or sorbitol.

In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR) 1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises a heavy chain (HC) comprising the amino acid sequence as set forth in SEQ ID NO: 12 and a light chain (LC) comprising the amino acid sequence as set forth in SEQ ID NO: 13. In some aspects, the anti-APRIL antibody comprises zigakibart/BION-1301.

In some aspects, the pharmaceutical composition is a liquid formulation. In some aspects, the pharmaceutical composition is a liquid formulation for subcutaneous injection. In some aspects, the pharmaceutical composition is contained within a syringe. In some aspects, the pharmaceutical composition is contained within an autoinjector. In some aspects, the pH of the pharmaceutical composition is between about 5 and about 7, e.g., about 5.0, about 5.25, about 5.5, about 5.75, about 6.0, about 6.25, about 6.5, about 6.75, or about 7.0. In some aspects, the pH is about 6.2. In some aspects, the total volume of the composition is between about 1 and about 3 mL, e.g., about 1 mL, about 1.25 mL, about 1.5 mL, about 1.75 mL, about 2.0 mL, about 2.25 mL, about 2.5 mL, about 2.75 mL, or about 3.0 mL. In some aspects, the total volume of the composition is about 2 mL.

TABLE 4
The ingredients of an exemplary pharmaceutical
composition are provided below.

| Ingredients | Mass (mg) | Concentration | |
|---|---|---|---|
| L-arginine | 17.6 | 51 | mM |
| L-glutamic acid | 14.8 | 50 | mM |
| L-histidine | 4.3 | 14 | mM |
| L-histidine | 4.6 | 11 | mM |
| monohydrochloride | | | |
| Polysorbate 80 | 0.4 | 200 | mg/mL |
| Sorbitol | 36.4 | 100 | mM |

In some aspects, the pharmaceutical composition comprises the ingredients described herein, e.g., the ingredients described in Table 4. In some aspects, the pharmaceutical composition comprises about 400 mg of the anti-APRIL antibody, about 17.6 mg of L-arginine, about 14.8 mg of L-glutamic acid, about 4.3 mg of L-histidine, about 4.6 mg of L-histidine monohydrochloride, about 0.4 mg of polysorbate 80, and about 36.4 mg of sorbitol. In some aspects, the pharmaceutical composition further comprises water for injection. In some aspects, the pH of the composition is about 6.2. In some aspects, the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol. In some aspects, the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol, and wherein the pH of the composition is about 6.2. In some aspects, the composition comprises about 200 mg/mL of the anti-APRIL antibody, about 51 mM L-arginine, about 50 mM L-glutamic acid, about 25 mM L-histidine, about 200 mg/mL polysorbate 80, and about 100 mM sorbitol. In some aspects, the pharmaceutical composition further comprises a trace amount of a chloride ion. In some aspects, the composition comprises between about 10 mg and about 1000 mg of the anti-APRIL antibody, e.g., about 10 mg, about 50 mg, about 100 mg, about 150 mg, about 200 mg, about 250 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 800 mg, about 900 mg, or about 1000 mg. In some aspects, the composition comprises about 400 mg of the anti-APRIL antibody. In some aspects, the pharmaceutical composition further comprises an overfill. In some aspects, the overfill comprises about 0.0125 mL to about 0.5 mL, about 0.025 mL to about 0.5 mL, about 0.05 mL to about 0.075 mL, about 0.075 mL to about 0.1 mL, about 0.1 mL to about 0.125 mL, about 0.125 mL to about 0.15 mL, about 0.15 mL to about 0.175 mL, about 0.175 mL to about 0.2 mL, about 0.2 mL to about 0.225 mL, about 0.225 mL to about 0.25 mL, about 0.25 mL to about 0.275 mL, about 0.275 mL to about 0.3 mL, about 0.3 mL to about 0.325 mL, about 0.325 mL to about 0.35 mL, about 0.35 mL to about 0.375 mL, about 0.375 mL to about 0.4 mL, about 0.4 mL to about 0.425 mL, about 0.425 mL to about 0.45 mL, about 0.45 mL to about 0.475 mL, or about 0.475 mL to about 0.5 mL. In some aspects, the overfill comprises about 0.0125 mL, about 0.025 mL, about 0.05 mL, about 0.075 mL, about 0.1 mL, about 0.125 mL, about 0.15 mL, about 0.175 mL, about 0.2 mL, about 0.225 mL, about 0.25 mL, about 0.275 mL, about 0.3 mL, about 0.325 mL, about 0.35 mL, about 0.375 mL, about 0.4 mL, about 0.425 mL, about 0.45 mL, about 0.475 mL, or about 0.5 mL.

In some aspects, the present disclosure provides a pharmaceutical composition comprising about 51 mM L-arginine, about 50 mM L-glutamic acid, about 14 mM L-histidine, about 11 mM L-histidine monohydrochloride, about 200 mg/mL polysorbate 80, about 100 mM sorbitol, and about 200 mg/mL of the anti-APRIL antibody, wherein the pH of the composition is about 6.2, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.

In some aspects, the present disclosure provides a pharmaceutical composition comprising about 51 mM L-arginine, about 50 mM L-glutamic acid, about 25 mM L-histidine, about 200 mg/mL polysorbate 80, about 100 mM sorbitol, and about 200 mg/mL of the anti-APRIL antibody, wherein the pH of the composition is about 6.2, wherein the total volume of the composition is about 2 mL, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody in the composition is about 400 mg. In some aspects, the anti-APRIL antibody comprises Sibeprenlimab or an antigen binding portion thereof.

In some aspects, the present disclosure provides a method of treating a disease or disorder associated with APRIL, e.g., IgA nephropathy or Sjögren's disease, in a subject in need thereof comprising administering to the subject the pharmaceutical composition.

In some aspects, the present disclosure provides a method of producing a pharmaceutical composition comprising admixing one or more ingredients in the pharmaceutical composition.

### EXAMPLES

### Example 1: Differential Impacts of APRIL and APRIL/BAFF on Immune Populations

### Background and Aims

APRIL and BAFF are essential cytokines for B cell and plasma cell development and survival. Inhibition of APRIL and APRIL/BAFF has emerged as a promising strategy for the treatment of immune-mediated diseases and kidney disorders, but their broader impacts on immune homeostasis are not fully known. Given the importance of B cells in immunity, such inhibition may pose risks of immunosuppression and susceptibility to infection. To address this, the differential effects of APRIL and APRIL/BAFF inhibition on immune cell populations, serological responses, and protective immunity were characterized in mouse models.

### Methods

### Flow Cytometry and Serology Assays

C57BL/6 mice were dosed twice per week with anti-APRIL antibody (4540), or APRIL/BAFF inhibitors (TACI-Fc fusion proteins POV and ATA, based on the sequences of povetacicept (ALPN-303) and atacicept (VT-001), respectively), or isotype control antibody based on the sequences of motavizumab (MOTA).

Serum samples were collected weekly, and immunoglobulin levels (IgA, IgM, and IgG) were quantified by ELISA (Bethyl Lab Kits).

Eight (8) weeks after initial dosing, mice were sacrificed, and spleens were processed to single cells prior to cryopreservation. To characterize B and T cell populations, immunophenotyping was performed on splenocytes. Cells were analyzed on a Sony ID 7000 spectral analyzer. B- and T-cell populations were identified using the following gating schemes

### (Table 5):

TABLE 5
Gating Schemes

| Cell Population | Gating Scheme Markers |
|---|---|
| PB | CD45+ CD19+ CD3/11b/14/56− B220+ CD138+ MHC II+ IgM+ (or IgM− |
| | IgA/G1/G2+ for CSw) |
| PC | CD45+ CD19− CD3/11b/14/56− B220− CD138+ MHC II− IgM+ (or IgM− |
| | IgA/G1/G2+ for CSw) |
| T1 (POV) | CD45+ CD19+ CD3/11b/14/56− B220+ CD138(Synd1)− CD23− CD21/35− |
| | IgM+ |
| T1 (BD) | CD45+ CD19+ CD3/11b/14/56− B220+ CD93+ CD23− IgM+ |
| T2 (POV) | CD45+ CD19+ CD3/11b/14/56− B220+ CD138(Synd1)− CD23+ |
| | CD21/35+ IgM+ |
| T2 (BD) | CD45+ CD19+ CD3/11b/14/56− B220+ CD93+ CD23+ IgM+ |
| T3 (BD) | CD45+ CD19+ CD3/11b/14/56− B220+ CD93+ CD23+ IgM− |
| MZ (POV) | CD45+ CD19+ CD3/11b/14/56− B220+ CD138(Synd1)− CD23− CD21/35+ |
| | IgM+ |
| MZ (BD) | CD45+ CD19+ CD3/11b/14/56− B220+ CD93− CD23− CD21/35+ |
| Follicular (POV) | CD45+ CD19+ CD3/11b/14/56− B220+ CD138(Synd1)− CD23+ |
| | CD21/35+ |
| Follicular (BD) | CD45+ CD19+ CD3/11b/14/56− B220+ CD93− CD23+ CD21/35+ |
| GC | CD45+ CD19+ CD3/11b/14/56− B220+ GL7+ CD95+ |
| Activated B | CD45+ CD19+ CD3/11b/14/56− B220+ CD138(Synd1)− CD80+ PDL2− |
| | MHC II+ IgM+ IgD− |
| Mem B | CD45+ CD19+ CD3/11b/14/56− CD138(Synd1)− IgD− IgM− |
| CSw Mem B | CD45+ CD19+ CD3/11b/14/56− CD138(Synd1)− IgD− IgM− CD38+ |
| IgA/G1/G2+ | CD45+ CD19+ CD3/11b/14/56− CD138(Synd1)− IgD− IgM− CD38+ |
| MemB | IgA/G1/G2+ |
| B1a | CD45+ CD19+ CD3/11b/14/56− B220low CD43+ CD5+ |
| B1b | CD45+ CD19+ CD3/11b/14/56− B220low CD43+ CD5− |
| Naïve T | CD45+ CD4/19+ TCRb+ CD44− CD62L+ |
| Tem | CD45+ CD4/19+ TCRb+ CD44+ CD62L− |
| Tcm | CD45+ CD4/19+ TCRb+ CD44+ CD62L+ |
| Treg | CD45+ CD4/19+ TCRb+ CD25+ CD127− |
| Teff | CD45+ CD4/19+ TCRb+ CD25+ CD127+ |
| ICOS CD44 | CD45+ CD4/19+ TCRb+ CD25+ CD127− CD44+ CD278(ICOS)+ |
| Treg | |
| Tfh | CD45+ CD4/19+ TCRb+ CXCR5+ PD1+ CD278(ICOS)+ |
| Tfr | CD45+ CD4/19+ TCRb+ CXCR5+ PD1+ CD278(ICOS)+ CD25+ CD127− |

### Single Cell RNA Sequencing and Spatial Transcriptomic Analysis

Single cell transcriptomics and surface proteomics data were obtained by cellular indexing of transcriptomes and epitopes by sequencing (CITE-Seq). Cells isolated from the spleen of treated mice were stained with Biolegend Universal Mouse Cocktail TotalSeq-C V1.0 supplemented with additional antibodies directed to CD273, CD80, CD95, IgM, CD267, CD319, IgG2b, and IgG1. Libraries were subsequently generated using 10× Genomics GEM-X Single Cell 5′ kit V3 following the manufacturer's protocol and sequenced by Illumina NovaSeq technology. Spatial transcriptomics data were generated from spleens of treated mice. Spleens were formalin fixed and paraffin embedded. Sections of 5 μm depth were used for data generation. 10× Genomics Xenium technology with the 5K Mouse Panel following manufacturer's protocol. Data was analyzed with single cell and spatial bioinformatics software (Seurat and Squidpy bioinformatics software packages).

### Results

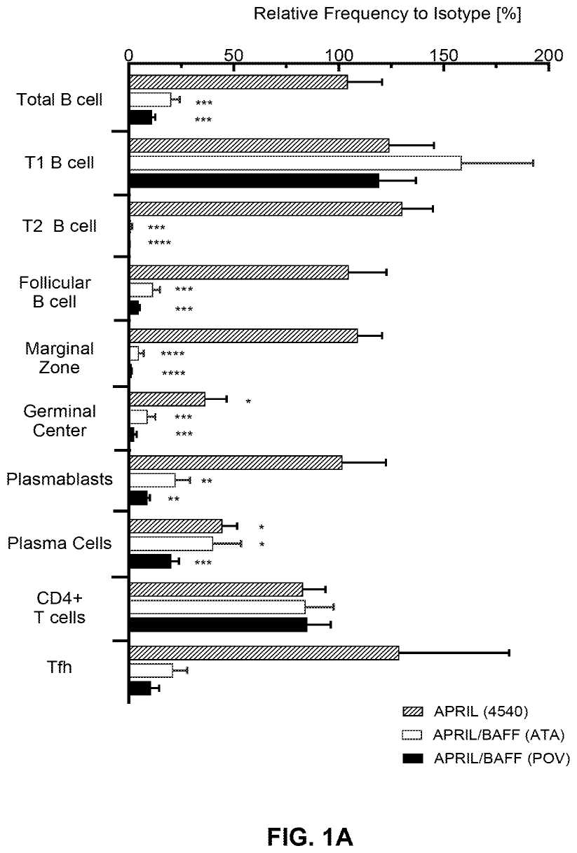

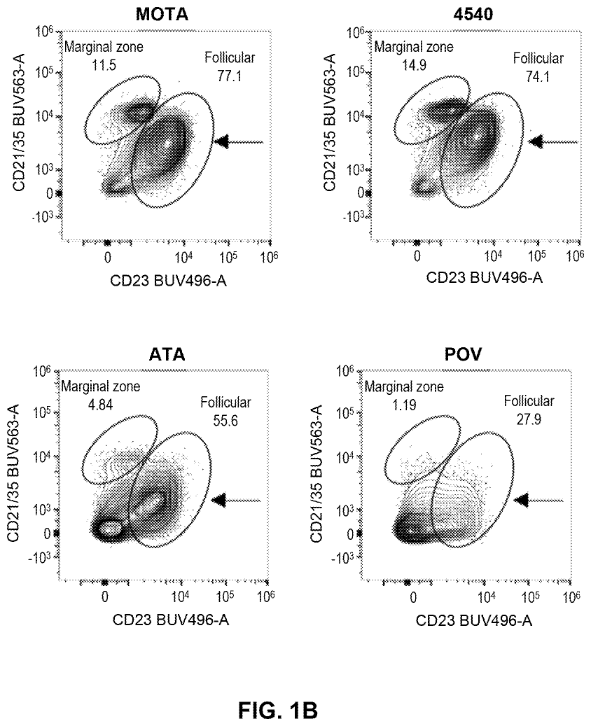

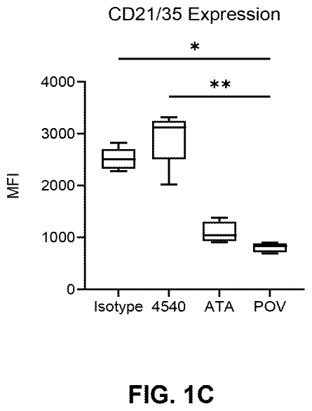

Repeated dosing of mice with dual APRIL/BAFF inhibitors led to a reduction of multiple B cell subsets, including immature, intermediate, matured, activated, and terminally differentiated populations (FIG. 1A). Total B cells were reduced by 89% and 80%. Follicular B cell populations were reduced by dual APRIL/BAFF inhibitors and exhibited lower CD21/35 expression (FIG. 1B and FIG. 1C), suggesting an abrogation of maturation and proliferation potential. Conversely, APRIL-only inhibition did not cause a reduction in total B cells, and only germinal center B cells and plasma cells decreased from treatment (FIG. 1A). Plasmablasts were significantly reduced with POV or ATA treatment, whereas APRIL-only inhibition did not impact abundance of that population (FIG. 1A).

APRIL and APRIL/BAFF inhibition had minimal impact on most T cell populations. APRIL/BAFF dual inhibitors, but not anti-APRIL antibody, led to a reduction in T follicular helper (Tfh) cells by 89%, an observation consistent with BAFF promoting development of Tfh cells (FIG. 1A).

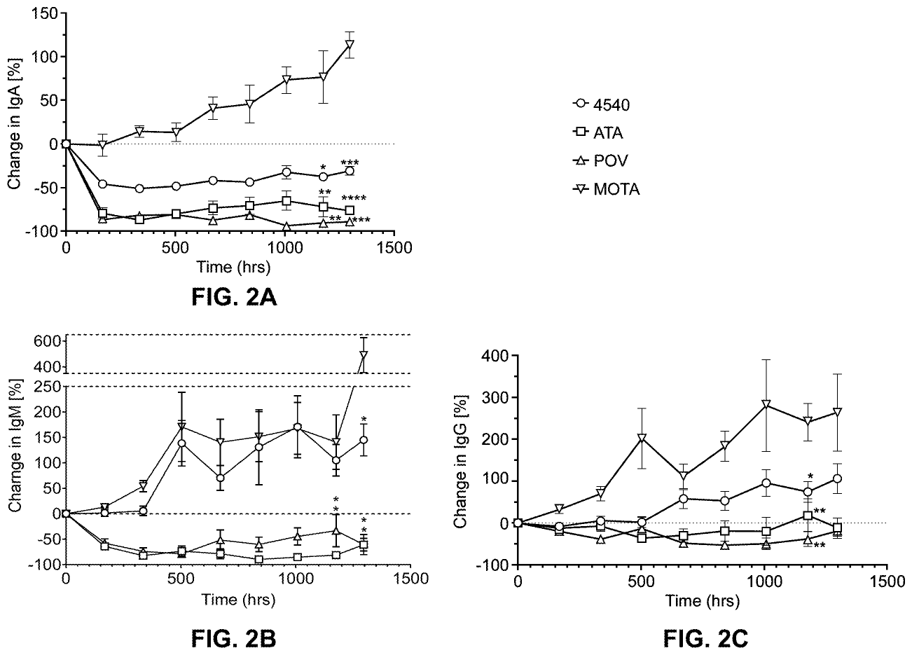

Serology measurements from the same treated mice demonstrated both classes of inhibitors yielded ≥40% reduction in IgA relative to pre-treatment. The APRIL/BAFF inhibitors also reduced serum IgM and IgG by ≥60% and ≥20% relative to pre-treatment, respectively, while APRIL-only did not reduce IgM or IgG (FIGS. 2A-2C). The implication of these immune cell changes to protective immunity against respiratory viruses is thus presented within the mouse.

### CONCLUSIONS

This study demonstrates that administration of both APRIL and APRIL/BAFF inhibitors reduce serum IgA, a desirable effect for kidney diseases like IgA nephropathy, in which elevated levels of aberrantly glycosylated IgA have a pathogenic role. However, dual APRIL/BAFF inhibition leads to significant ablation of most B cell subsets and antibody-secreting cells, whereas, unexpectedly, APRIL-only inhibition does not substantially alter abundance of most splenic B cell populations. This broad ablation of B cells has implications for immune homeostasis and B cell responses to pathogens during infection.

### Example 2: Influenza Challenge of APRIL or APRIL/BAFF Treated C57BL/6 Mice

### Background and Aims

Mice treated with APRIL or APRIL/BAFF inhibitors as well as inactivated influenza virus vaccine were challenged with Cal09 influenza virus to assess the differential effects of APRIL and APRIL/BAFF inhibition on infection susceptibility.

### Methods

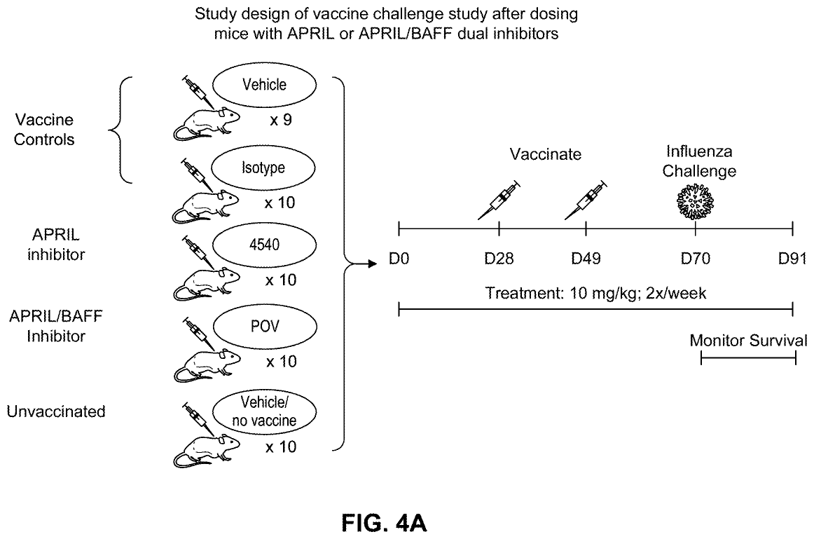

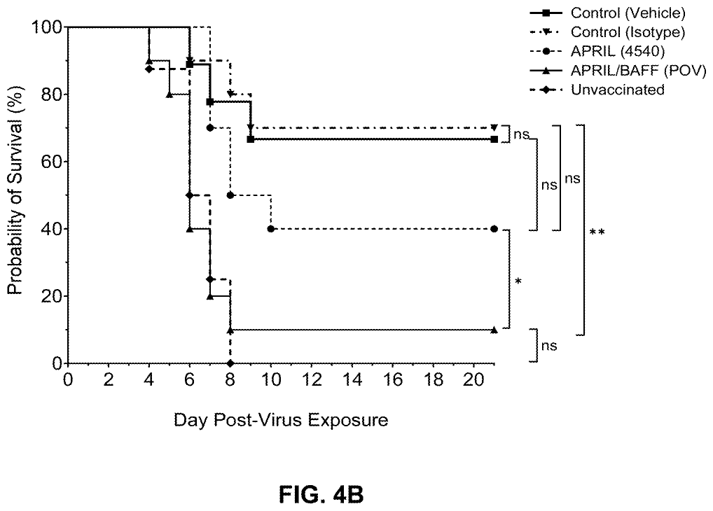

Mice were dosed twice per week with anti-APRIL antibody (4540; SEQ ID Nos: 12 and 13), or APRIL/BAFF inhibitors (TACI-Fc fusion proteins POV and ATA, based on the sequences of povetacicept (ALPN-303) and atacicept (VT-001), respectively; SEQ ID Nos. 14 and 15, respectively), or isotype control antibody based on the sequences of motavizumab (MOTA; SEQ ID Nos: 16 and 17) for the duration of the study. Two doses of inactivated influenza virus vaccine (5 μg total protein of BEI inactivated MDCK-cell adapted Cal09 strain) were administered to mice at week 4 (day 28) and week 7 (day 49). Animals were challenged with Cal09 influenza virus at week 10 (day 70). Animals were either: 1) monitored for 21 days for signs of disease with animals being humanely euthanized if body weight loss was >30%, (FIG. 4A). The probability of survival as a function of day post-virus exposure was estimated using Kaplan-Meier survival curves for each group (FIG. 4B).

### Results

Dual APRIL/BAFF inhibition, but not APRIL-only inhibition, compromised vaccine-mediated protection (FIG. 4B). Mice receiving APRIL/BAFF treatment had a lower probability of survival relative to isotype control (**p=<0.01, log-rank (Mantel-Cox)) and APRIL-only treatment (*p=<0.05, log-rank (Mantel-Cox)) (FIG. 4B). APRIL/BAFF treatment was not significantly different than the unvaccinated cohort (FIG. 4B).

### Example 3: Keyhole Limpet Hemocyanin (“KLH”) Vaccination of Mice with APRIL, BAFF, or APRIL/BAFF Treatment

### Background and Aims

Mice treated with APRIL, BAFF, or APRIL/BAFF inhibitors were vaccinated with KLH to assess the differential effects of APRIL, BAFF, and APRIL/BAFF inhibition on immune response.

### Methods

### Pre-Treatment Study Investigating Primary Response to KLH

Mice are dosed twice per week for 6 weeks at 10 mg/kg with anti-APRIL antibody (4540), anti-BAFF antibody (Sandy-2) or APRIL/BAFF inhibitors (TACI-Fc fusion proteins POV and ATA, based on the sequences of povetacicept (ALPN-303) and atacicept (VT-001), respectively), or isotype control antibody based on the sequences of motavizumab (MOTA). Animals receive 2 doses of KLH (250 μg each) at week 2 and week 4. Anti-KLH titers are quantified by ELISA at week 4 and 6 and compared to pre-study titers.

### Memory Re-Call Study

Mice were dosed with KLH (250 μg) on day 0 and day 42. Two weeks after initial dosing (day 14), mice were treated 2×/week with 10 mg/kg with anti-APRIL antibody (4540), APRIL/BAFF inhibitors (TACI-Fc fusion proteins POV, based on the sequences of povetacicept), or isotype control antibody (MOTA) for the duration of the study (through day 56). Serum KLH-specific IgM titers were measured on days 14 (pre-inhibitor), 42 (pre-KLH boosting), 49, and 56. KLH-specific IgG titers were assessed on days 14, 42, 49, and 56.

### Results

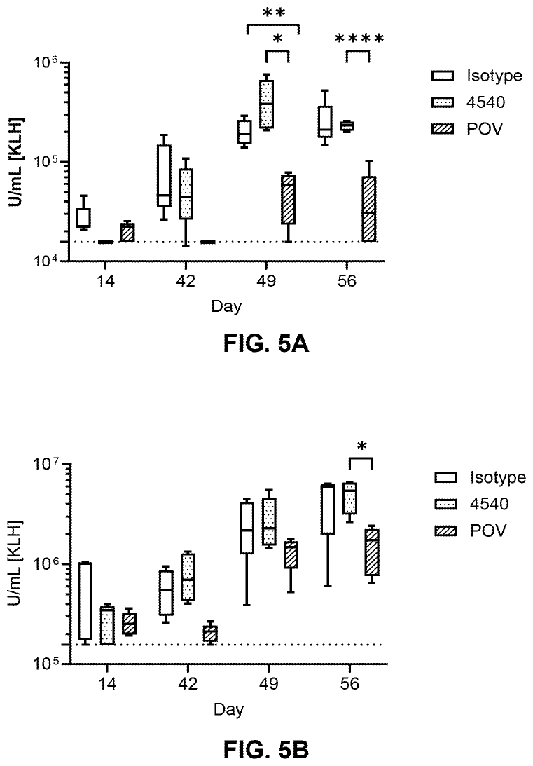

Dual APRIL/BAFF, but not APRIL-only inhibition, reduced vaccine serological responses (FIGS. 5A-5B). APRIL/BAFF inhibition resulted in a dampened or attenuated duration of IgM response compared with control, especially evident following day 42 (FIG. 5A). APRIL/BAFF inhibition also resulted in a delayed or dampened IgG response compared with control, especially evident following day 42 (FIG. 5B).

### Example 4: SARS-Cov2 Challenge of APRIL or APRIL/BAFF Treated BALB/C Mice

### Background and Aims

Mice treated with APRIL or APRIL/BAFF inhibitors challenged with SARS-Cov2 MA10 are assessed for the differential effects of APRIL and APRIL/BAFF inhibition on infection susceptibility.

### Methods

Mice are dosed twice per week with anti-APRIL antibody (4540), or APRIL/BAFF inhibitors (TACI-Fc fusion proteins POV and ATA, based on the sequences of povetacicept (ALPN-303) and atacicept (VT-001), respectively), or isotype control antibody based on the sequences of motavizumab (MOTA) for the duration of the study. Pfizer mRNA CoVID vaccine (Comirnaty) are administered to mice. Animals are subsequently challenged with mouse adapted strain of SARS-Cov. Animals are monitored for 15 days for signs of disease. Animals are humanely euthanized if body weight loss is >30%. Additionally, neutralizing antibody titer after CoVID vaccination is measured in a Luminex-based assay. Briefly, biotinylated human ACE2 and vaccinated mouse sera are pre-incubated. Binding of ACE2 to CoVID spike protein is measured, with neutralizing antibody preventing interaction of ACE2/spike. Neutralizing titer is measured after CoVID vaccine to assay the impact of treatment on vaccine response and is correlated to protection in the virus challenge model.

### Example 5: Effect of VIS649 IV Administration on Lymphocytes

### Background and Aims

This was a Phase 2 multicenter, randomized, double-blind, placebo-controlled, multiple-dose study to evaluate the safety and efficacy of IV administered VIS649 over a 12-month treatment course in participants with IgAN. The effect of VIS649 IV administration on lymphocytes was assessed as an exploratory endpoint.

### Methods

Participants were enrolled from 98 centers across 15 countries. Enrolled participants were 18 years of age or older with an IgAN diagnosis confirmed by biopsy. VIS649 (2, 4, and 8 mg/kg) or placebo was administered via 12 monthly IV infusions. The exploratory B lymphocyte populations shown in the B Cell Class Switch Panel (Table 6) were examined at Month 0, 6, 12, and 16. Lymphocytes in whole blood and change from baseline over time were analyzed on a flow cytometer (Becton Dickinson, SORP FACSCanto™ II).

TABLE 6
B Cell Class Switch Panel

| Population | Unit |
|---|---|
| % Lymphs, CL | % |
| % MNC, CL | % |
| % CD19+ (Lymphs), CL | % |
| % CD19+ (MNC), CL | % |
| %38dm− (19+/IgD+/27−) Ly, CL | % |
| % CD27+ (CD19+ Lymphs), CL | % |
| % IgD+/CD27+ (CD19+ Ly), CL | % |
| % IgD−/CD27+ (CD19+ Ly), CL | % |
| % IgD−/CD27− (CD19+ Ly), CL | % |
| %38br (19+/IgD+/27−) MNC, CL | % |
| %38br (CD19+/CD27+) MNC, CL | % |
| % CD27+/CD43+ (CD19+ Ly), CL | % |
| % CD27+/CD43− (CD19+ Ly), CL | % |
| % IgM+/IgD+ (19+/27+/43−), CL | % |
| % IgM+/IgD− (19+/27+/43−), CL | % |
| % IgA+ (CD19+ Lymphs), CL | % |
| % IgA+ (CD19+/CD27+) Ly, CL | % |
| % IgA+ (19+/27+/IgD−) Ly, CL | % |
| % IgA+ (19+/27+/38br) MNC, CL | % |
| % IgM+ (CD19+ Lymphs), CL | % |
| % IgM+ (CD19+/CD27+) Ly, CL | % |
| % IgM+ (19+/27+/IgD−) Ly, CL | % |
| % IgM+ (19+/27+/38br) MNC, CL | % |
| MESF IgA+ (CD19+ Lymphs), CL | MESF |
| MESF IgA+ (19+/CD27+) Ly, CL | MESF |
| MESF IgA+(19+27+IgD−) Ly, CL | MESF |
| MESF IgA+(19+27+38br)MNC, CL | MESF |
| MESF IgM+ (CD19+ Lymphs), CL | MESF |
| MESF IgM+ (19+/CD27+) Ly, CL | MESF |
| MESF IgM+(19+27+IgD−) Ly, CL | MESF |
| MESF IgM+(19+27+38br)MNC, CL | MESF |
| Abs Lymphs, CL | cells/μL |
| Abs MNC, CL | cells/μL |
| Abs CD19+ (Lymphs), CL | cells/μL |
| Abs CD19+ (MNC), CL | cells/μL |
| Abs 38dm− (19+ID+27−) Ly, CL | cells/μL |
| Abs CD27+ (CD19+ Lymphs), CL | cells/μL |
| Abs IgD+/27+ (CD19+ Ly), CL | cells/μL |
| Abs IgD−/27+ (CD19+ Ly), CL | cells/μL |
| Abs IgD−/27− (CD19+ Ly), CL | cells/μL |
| Abs 38br (19+IgD+27−)MNC, CL | cells/μL |
| Abs 38br (19+/27+) MNC, CL | cells/μL |
| Abs 27+/CD43+ (CD19+ Ly), CL | cells/μL |
| Abs 27+/CD43− (CD19+ Ly), CL | cells/μL |
| Abs IgM+IgD+ (19+27+43−), CL | cells/μL |
| Abs IgM+IgD− (19+27+43−), CL | cells/μL |
| Abs IgA+ (CD19+ Lymphs), CL | cells/μL |
| Abs IgA+ (19+/CD27+) Ly, CL | cells/μL |
| Abs IgA+ (19+27+IgD−) Ly, CL | cells/μL |
| Abs IgA+(19+27+38br) MNC, CL | cells/μL |
| Abs IgM+ (CD19+ Lymphs), CL | cells/μL |
| Abs IgM+ (CD19+CD27+) Ly, CL | cells/μL |
| Abs IgM+ (19+27+IgD−) Ly, CL | cells/μL |
| Abs IgM+ (19+27+38br)MNC, CL | cells/μL |

Abs = antibodies;
MNC = mononuclear cell;
Lymphs = Lymphocytes;
Ig = Immunoglobulin,
MESF = Molecules of Equivalent Soluble Fluorochrome;
CL = complete lymphocytes;
Ly =

### Results

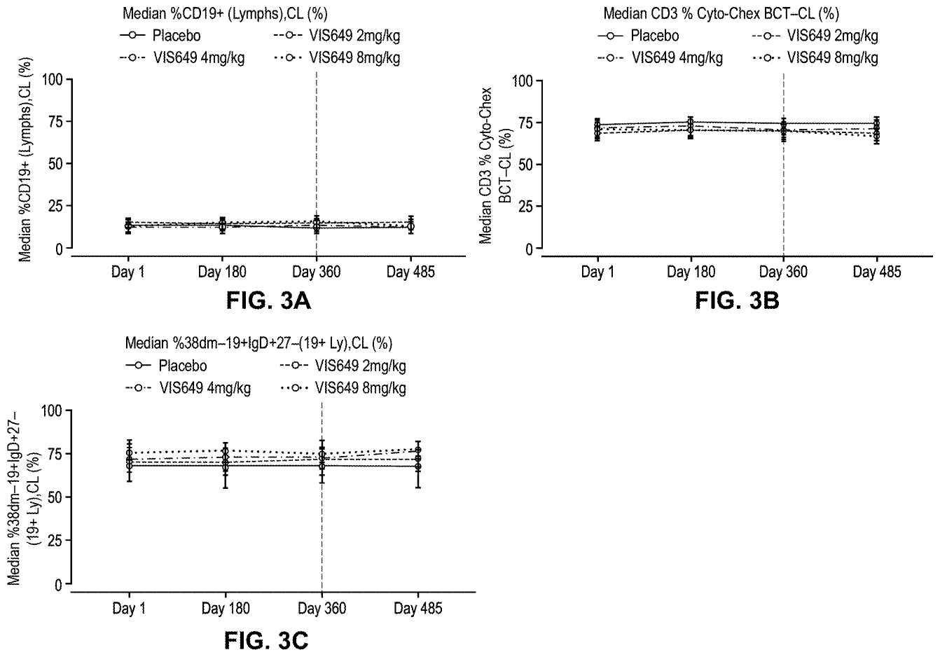

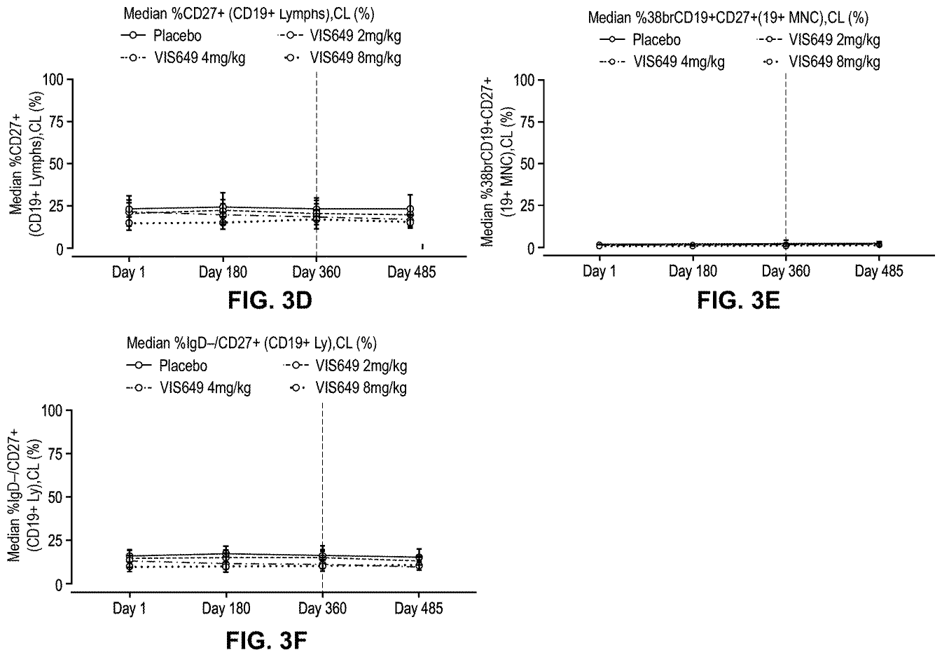

As shown in FIGS. 3A-3F, total B cells, naïve B cells, total memory cells, switched memory B cells, and plasmablasts showed no significant modulations over 12 months of sibeprenlimab treatment.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific aspects of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1.-22. (canceled)

23. A method of providing an anti-APRIL therapy for treating an IgA nephropathy or autoimmune disease in a subject in need thereof (a) identifying a subject who needs a treatment for an IgA nephropathy or autoimmune disease and has a risk of an infection during the treatment, (b) selecting, among anti-APRIL therapies comprising an anti-APRIL antibody and an APRIL/BAFF inhibitor, an anti-APRIL antibody as an anti-APRIL therapy suitable for the subject, and (c) administering the anti-APRIL antibody to the subject.

24. The method of claim 23, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR)1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8.

25. The method of claim 23, wherein the VH comprises the amino acid sequence as set forth in SEQ ID NO: 1 and the VL comprises the amino acid sequence as set forth in SEQ ID NO: 2.

26.-30. (canceled)

31. The method of claim 30, wherein the APRIL/BAFF inhibitor comprises a fusion protein.

32. The method of claim 23, wherein the APRIL/BAFF inhibitor comprises atacicept, povetacicept, or telitacicept.

33.-40. (canceled)

41. The method of claim 23, wherein the subject does not have a severe and/or active infection.

42. The method of claim 23, wherein the subject has a mild infection or a moderate infection.

43. (canceled)

44. (canceled)

45. A method of identifying a subject suitable for the treatment of an anti-APRIL therapy comprising (a) assessing the subject's risk of infection during the treatment and (b) selecting an anti-APRIL antibody as a suitable treatment if the subject has a risk of infection during the treatment.

46. (canceled)

47. (canceled)

48. The method of claim 46, wherein the subject is in need of treatment for an IgA nephropathy or autoimmune disease.

49.-70. (canceled)

71. A method of reducing or preventing a risk of infection in a subject in need of an anti-APRIL therapy comprising administering to the subject an anti-APRIL antibody.

72. (canceled)

73. The method of claim 71, wherein the subject is in need of treatment for an IgA nephropathy or autoimmune disease.

74.-95. (canceled)

96. A method of treating an IgA nephropathy or autoimmune disease in a subject in need thereof comprising administering to the subject an anti-APRIL antibody, wherein the subject is identified as having a risk of infection during the treatment.

97. A method of treating an IgA nephropathy or autoimmune disease in a subject in need thereof comprising administering to the subject an anti-APRIL antibody after the subject has developed an infection.

98.-120. (canceled)

121. A method of maintaining B cell population during an anti-APRIL therapy in a subject in need of the therapy comprising administering to the subject an anti-APRIL antibody.

122.-134. (canceled)

135. The method of claim 23, wherein the administering does not significantly alter the B cell population compared to an APRIL/BAFF inhibitor.

136. The method of claim 23, wherein the administering does not reduce IgM and/or IgG levels in the subject.

137. The method of claim 23, wherein the infection comprises a viral infection, a bacterial infection, a fungal infection, or a parasitic infection.

138. The method of claim 23, wherein the infection causes an infectious disease.

139. The method of claim 23, wherein the autoimmune disease comprises rheumatoid arthritis, systemic lupus erythematosus, a linear IgA bullous disease, IgA-mediated epidermolysis bullosa acquisita, or Sjögren's disease.
